(19) United States
(12) Patent Application Publication
(10) Pub. No.: US 2008/0020440 A1
(43) Pub. Date: Jan. 24, 2008

(54) Method of sequestering and/or purifying a polypeptide

(76) Inventors: Daniel Tillett, Sydney (AU); Thomas Torsten, Coogee (AU)

Correspondence Address: PROTIGENE PTY LTD, LEVEL 9, BARRACK HOUSE, 16- 20 BARRACK STREET, SYDNEY, 2000, AU

(21) Appl. No.: 10/785,452

(22) Filed: Feb. 25, 2004

(30) Foreign Application Priority Data
Aug. 27, 2002 (AU) PCT/AU02/01159
Aug. 27, 2002 (AU) 2002322186

(51) Int. Cl. C12N 1/00; C07H 21/00; C07K 1/14; C12N 15/63; C07K 14/00; C07K 2/00

(52) U.S. Cl. 435/243; 435/267; 435/320.1; 530/300; 530/350; 530/356; 530/357; 530/363; 530/367; 530/382; 530/385; 530/387.1; 530/395; 530/399; 530/400; 530/412; 536/23.1

(57) ABSTRACT

The present invention relates to DNA and polypeptide constructs and related methods useful for sequestering and/or purifying polypeptides. In particular, the invention relates to hybrid polypeptides comprising a polypeptide of interest linked to a polymerisable polypeptide, a method of sequestering and/or purifying a polypeptide of interest using the hybrid polypeptide and related hybrid nucleic acids, transformed cells and libraries.

### FIELD OF THE INVENTION

The present invention relates to DNA and polypeptide constructs and related methods useful for sequestering and/or purifying polypeptides. In particular, the invention relates to hybrid polypeptides comprising a polypeptide of interest linked to a polymerisable polypeptide, a method of sequestering and/or purifying a polypeptide of interest using the hybrid polypeptide and related hybrid nucleic acids, transformed cells and libraries.

### BACKGROUND OF THE INVENTION

Any discussion of the prior art throughout the specification should in no way be considered as an admission that such prior art is widely known or forms part of common general knowledge in the field.

It is currently possible to employ micro-organisms, capable of rapid and abundant growth, for the synthesis of commercially useful proteins and peptides. Such techniques make it possible to genetically endow a suitable micro-organism with the ability to synthesise a protein or peptide normally made by another organism (Makrides, 1996). In brief, DNA fragments coding for the protein are ligated into a cloning vector such as a plasmid. An appropriate host is transformed with the cloning vector and the transformed host is identified, isolated and cultivated to promote expression of the desired protein. Proteins so produced are then isolated from the culture medium for purification.

Many purification techniques have been employed to harvest the proteins produced by recombinant DNA techniques (Kornberg, 1990). Such techniques generally include segregation of the desired protein based on its distinguishing molecular properties, e.g. by dialysis, density-gradient centrifugation and liquid column chromatography. Such techniques are not universally applicable and often result in consumption of purification materials which may have considerably more commercial value than the protein being purified, particularly where substantial quantities of highly purified protein are desired (Ostove, 1990).

Other procedures have been developed to purify proteins based on the solubility characteristics of the protein. For example, isoelectric precipitation has been employed to purify proteins since the solubility of proteins varies as a function of pH. Similarly, solvent fractionation of proteins is a technique whereby the solubility of a protein varies as a function of the dielectric constant of the medium. Solvent fractionation, while giving good yields, often causes denaturation of the protein molecule. Neither isoelectric precipitation nor solvent fractionation are useful for obtaining highly purified protein. Such techniques are typically employed in tandem with other procedures.

Proteins have also been separated based on their ionic properties e.g. by electrophoresis, ion-exchange chromatography, etc. However, high purity and yield of the protein obtainable by such techniques is rarely achieved in a single step.

Affinity chromatography has also been employed in the purification of bio-polymers such as proteins (Ostove, 1990). Affinity chromatography involves placing a selective adsorbent in contact with a solution containing several kinds of substances including the desired species to be purified. For example, when used in protein purification protocols, affinity chromatography generally involves the use of a ligand which specifically binds to the protein to be purified. In general, the ligand is coupled or attached to a support or matrix and the coupled ligand is contacted with a solution containing the impure protein. The non-binding species are removed by washing and the desired protein recovered by eluting with a specific desorbing agent. While affinity chromatography produces a relatively high level of purified protein, this technique requires significant amounts of the protein-specific ligand employed for purification (Ostove, 1990). Moreover, the ligand will be different for each and every protein to be purified which necessarily entails a time-consuming and laborious regime. In addition, it has been found that specific ligands do not exist for all types of protein molecules, such as certain enzymes. As a result, affinity chromatography has not been successfully employed as a universal isolation purification technique for protein molecules.

One way to circumvent this problem is to construct a gene fusion between the open reading frame of the protein of interest, and an open reading frame encoding a specific ligand-binding domain. In this method the protein is produced, with an affinity tail, from a chimeric gene. Ligand-binding domain affinity tails that have been used successfully on a large number of different proteins including; oligohistidine (Arnold, 1991; Van Dyke et al., 1992), glutathione S-transferase (GST) (Smith & Johnson, 1988), and carbohydrate binding proteins (di Gaun et al., 1988; Taylor & Drickamer, 1991). For example, the frequently used oligohistidine method relies on the observation that oligohistidine is an excellent chelator of divalent metals ions such as Ni⁺² (Arnold, 1991; Van Dyke et al., 1992). A protein containing a sequence of six histidine residues either at the amino- or carboxyl-terminus of the protein has the capacity to bind, very strongly, to Ni⁺² (Van Dyke et al., 1992). Affinity chromatography using nickel linked to agarose is highly specific because such terminal 6×His-tag sequences do not occur naturally in E. coli. A crude cell lysate containing overexpressed 6×His-tagged protein can be added directly to an Ni⁺²-agarose column. Following the wash steps the 6×His-tagged protein is eluted from the column using imidazole, a competitor for Ni⁺² chelation.

However, methods that rely on affinity chromatography suffer from at least one major disadvantage: the sample must be exposed to affinity chromatographic media either in batch or in columns. This problem becomes an important consideration upon scale up of sample size or sample number.

It is an object of the present invention to overcome or ameliorate at least one of the disadvantages of the prior art, or to provide a useful alternative.

### SUMMARY OF THE INVENTION

It has surprisingly been found that polymerisable polypeptides can be used to sequester and/or purify polypeptides of interest.

Accordingly, in a first aspect, the present invention provides a hybrid polypeptide comprising a polypeptide of interest linked to a polymerisable polypeptide.

It will be clear to the skilled addressee that the polypeptide of interest may be linked by any suitable means to the polymerisable polypeptide. The linkage may be a direct linkage between the two polypeptides or may be made by means of a linker. Further, the polypeptides may be linked by covalent or non-covalent bonds.

For example, the polypeptide of interest may be fused directly to the polymerisable polypeptide. Alternatively, and in a preferred embodiment, the polypeptide of interest is linked to the polymerisable polypeptide by a linker polypeptide.

Preferably, the hybrid polypeptide is produced in vivo.

When the polypeptide of interest is linked to the polymerisable polypeptide by a linker polypeptide, the linker polypeptide may be introduced into the hybrid polypeptide between the polypeptide of interest and the polymerisable polypeptide in vivo or in vitro. The linker polypeptide may be introduced into the hybrid polypeptide by recombinant DNA techniques. The polypeptide of interest, linker polypeptide and polymerisable polypeptide may be produced from one nucleic acid molecule encoding the hybrid polypeptide and the nucleic acid may be introduced into an expression system to produce the hybrid polypeptides in large quantities.

The linker polypeptide is preferably encoded by a polynucleotide comprising a recognition site for a proteolytic agent. In a preferred embodiment, the linker polypeptide comprises the recognition site for 3C-protease from human rhinovirus type 14 (HRV protease 3C), Leu-Glu-Val-Leu-Phe-Gln-Gly-Pro. HRV protease 3C cleaves this recognition site between the glutamine and glycine residues. The skilled addressee will recognise that for some applications, it is desirable that the cleavage be close to, or at, the amino acid defining the end of the polypeptide of interest particularly if additional amino acids are likely to interfere with the function of the polypeptide of interest.

It will be clear to the skilled addressee that linker polypeptides comprising recognition sites for other proteases such as thrombin (recognition sequence Leu-Val-Pro-Arg-Gly-Ser), Factor Xa (recognition sequence Ile-Glu-Gly-Arg) or enterokinase (recognition sequence Asp-Asp-Asp-Asp-Lys) will also be useful in the present invention.

Preferably, the linker polypeptide comprises a cloning site and most preferably the cloning site is a multiple cloning site. It will be clear to the skilled addressee that the multiple cloning site may itself include, or consist of, the recognition site for cleavage by the protease. In certain constructs, the linker may comprise, or consist of, a spacer polypeptide of sufficient length to allow or enhance cleavage of the polypeptide of interest from the polymerisable polypeptide i.e. by allowing access to a proteolytic enzyme for cleavage of the hybrid polypeptide, or to avoid unfavourable steric interference between the polypeptide of interest and the polymerisable polypeptide.

The linker polypeptide may also be a recognition site for chemical cleavage e.g. cyanogen bromide or self-cleavable protein elements such as inteins.

As a further alternative, the polypeptides may be linked by antibody interaction e.g. by attaching an antibody specific for the polymerisable polypeptide to the polypeptide of interest or, alternatively, by attaching an antibody specific for the polypeptide of interest to the polymerisable polypeptide. As a further alternative, the linker may be a bi-specific antibody directed to both the polypeptide of interest and the polymerisable polypeptide.

Preferably, the polymerisable polypeptide is a polypeptide that naturally polymerises with itself (self-polymerising). More preferably the polymerisable polypeptide is a tubulin or actin. Most preferably it is an FtsZ polypeptide or variant thereof and, in a preferred embodiment, the polymerisable peptide is E. coli FtsZ polypeptide or a variant thereof. However, it will be clear to the skilled addressee that any polymerisable polypeptide may be used and that polypeptides which require an intermediary polypeptide or other molecule in order to polymerise (as opposed to self-polymerising polypeptides) are also contemplated.

Variants of FtsZ having autopolymerisation and/or ATPase properties may also be suitable as the polymerisable polypeptide in the present invention. For example, a modified FtsZ may be polymerised by addition of ATP and divalent cations. A mutant FtsZ polymerisable polypeptide may be used which, due to replacement of the aspartate residue at position 212 of the E. coli FtsZ with a cysteine or asparagine residue, allows for polymerisation using a cation such as magnesium, calcium, nickel, cobalt, zinc or manganese. Alternatively, a variant form of the E. coli FtsZ polypeptide which comprises mutations at positions 70 (alanine to threonine), 209 (aspartate to alanine) or 269 (aspartate to alanine) may also be employed in the present invention since the polymerisation properties of the polypeptide are retained.

It will be clear to the skilled addressee that the polypeptide of interest may be of any origin, including prokaryotic or eukaryotic origin and may be a simple or a conjugated polypeptide. For example, the polypeptide of interest may be an endonuclease, a methylase, an oxidoreductase, a transferase, a hydrolase, a lysase, an isomerase or a ligase. The polypeptide of interest may also be a storage polypeptide such as a ferritin or ovalbumin or a transport protein such as haemoglobin, serum albumin or ceruloplasmin. It may also be, for example, an antigen or antigenic determinant for use in the preparation of vaccines or diagnostic agents, a protective or defence protein, such as the blood protein thrombin, fibrinogen, a binding protein, an antibody or an immunoglobulin. The polypeptide of interest may also be, for example, a human growth hormone, somatostatin, prolactin, estrone, progesterone, melanocyte, thyrotropin, calcitonin, gonadotropin or insulin or a hormone identified as being involved in the immune system such as interleukin 1, interleukin 2, colony simulating factor, macrophage-activating factor and interferon. Further, the polypeptide of interest may be a structural element such as collagen, elastin, alpha-keratin, glyco-protein, virus-protein or muca-protein.

In certain embodiments, the polypeptide of interest may be a protease and in one or more embodiments it is preferably a 3C protease from human rhinovirus type 14.

It will be clear that the polypeptide of interest may also be a synthetic polypeptide of interest.

According to a second aspect, the present invention provides a method of sequestering and/or purifying a polypeptide of interest comprising the step of polymerising a hybrid polypeptide, which hybrid polypeptide comprises the polypeptide of interest linked to a polymerisable polypeptide.

Preferably, polymerisation is performed under controlled chemical and/or physical conditions.

In one or more embodiments, the polymerisable polypeptide is polymerised by addition of an agent which induces polymerisation. Preferably, the polymerisation inducing agent is GTP, ATP and/or a cation. More preferably, the cation is selected from the following group: magnesium, calcium, nickel, cobalt, zinc and manganese.

It will be clear to the skilled addressee that the polymerisable polypeptide may, in certain instances, be polymerised by a change in temperature either alone or in combination with the addition of a polymerisation inducing agent.

The polymerised hybrid polypeptide may be purified by a first purification step and, for some applications, the first purification step may be the only purification step required. Alternatively, the first purification step may be followed by further purification steps.

Preferably, the first purification step purifies the polymerised hybrid polypeptide by physical techniques discriminating on the basis of size and/or weight. More preferably, the polymerised hybrid polypeptide is purified by centrifugation by varying the rotational force and/or time such that the polymerised hybrid polypeptide is isolated. However, the skilled addressee will be aware that other methods of purification are also contemplated including, but not limited to, differential sedimentation, filtration, dialysis and flow sorting. These methods are common practice in the field of the invention.

Preferably, after the first purification step the polymerised hybrid polypeptide is dissociated. More preferably, dissociation is achieved by removal of the agent which induces polymerisation eg. GTP, ATP and/or cation (as appropriate), and/or incubation of the polymerised hybrid polypeptide at a suitable temperature. The conditions will vary depending on the polymerisable polypeptide and it is well within the competence of the skilled addressee to determine the appropriate conditions.

Most preferably, the dissociated hybrid polypeptide is purified by a second purification step and in a preferred embodiment the second purification step comprises purification of the hybrid polypeptide on the basis of size and/or weight.

Preferably, polymerisation, dissociation and purification of the polymerisable hybrid polypeptide are repeated such that substances larger and smaller than the hybrid polypeptide are removed.

Preferably, the polymerisable polypeptide is cleaved from the polypeptide of interest by a proteolytic agent. The skilled addressee will understand that it is preferable that the proteolytic agent does not substantially interfere with the biological or chemical activity of the polypeptide of interest or the polymerisable polype lptide.

Preferably, the proteolytic agent is a protease. Most preferably, the protease is 3C-protease from a human rhinovirustype 14 (HRV protease 3C).

Preferably, the protease is linked to a polymerisable polypeptide to form a “protease hybrid polypeptide”. This protease hybrid polypeptide can be prepared in the same way as the hybrid polypeptide of the invention. More preferably, the polymerisable polypeptide to which the protease is linked is identical to the polymerisable polypeptide to which the polypeptide of interest is linked, or is a variant thereof.

Preferably, after cleavage of the polypeptide of interest from the polymerisable polypeptide, the protease hybrid polypeptide is polymerised. More preferably, the polypeptide of interest is purified from the polymerised protease hybrid polypeptide. Most preferably, the polypeptide of interest is purified from the polymerised protease hybrid polypeptide by a method which discriminates on the basis of size and/or weight.

It will be clear to the skilled addressee that the proteolytic agent may also be fused or linked to the hybrid polypeptide. In a preferred embodiment, the proteolytic agent is fused or linked adjacent the polymerisable polypeptide. More preferably, the proteolytic agent is fused or linked such that it remains attached to the polymerisable polypeptide after cleavage of the protein of interest. It will be clear to the skilled addressee that polymerising the polymerisable polypeptide and the attached proteolytic agent would facilitate separation of the proteolytic agent from the polypeptide of interest.

Preferably, the polymerisable polypeptide released after cleavage from the polypeptide of interest is also polymerised. It will be clear to the skilled addressee that this may occur simultaneously with polymerisation of the protease hybrid polypeptide when such a protease is employed. More preferably, the polymerised polymerisable polypeptide is removed from the polypeptide of interest by a method which discriminates on the basis of size and/or weight.

In one or more embodiments, the hybrid polypeptide may be linked to a support. Preferably, the support comprises a polymerisable polypeptide. More preferably, the support polymerisable polypeptide comprises a polymerisable polypeptide identical to that of the hybrid polypeptide, or a variant thereof.

According to a third aspect, the present invention provides a hybrid nucleic acid comprising a nucleic acid encoding a hybrid polypeptide according to the first aspect.

According to a fourth aspect, the present invention provides a library comprising a plurality of hybrid nucleic acids according to the third aspect.

According to a fifth aspect, the present invention provides a vector comprising a hybrid nucleic acid according to the third aspect.

According to a sixth aspect, the present invention provides a library of vectors according to the fifth aspect.

According to a seventh aspect, the present invention provides a cell transformed or transfected with a hybrid nucleic acid according to the third aspect, a library according to the fourth aspect, a vector according to the fifth aspect or a library of vectors according to the sixth aspect.

According to an eighth aspect, the present invention provides cells transformed or transfected with a library according to the fourth or fifth aspect.

According to a ninth aspect, the present invention provides a library comprising a plurality of hybrid polypeptides according to the first aspect.

According to a tenth aspect, the present invention provides use of a hybrid nucleic acid according to the third aspect, a library of nucleic acids according to the fourth aspect, a vector according to the fifth aspect or a library of vectors according to the sixth aspect in a method of sequestering and/or purifying a polypeptide of interest according to the first aspect.

According to an eleventh aspect, the present invention provides a polypeptide of interest when purified by a method according to the second aspect.

According to a twelfth aspect, the present invention provides a library of polypeptides of interest when purified by a method according to the first aspect.

According to a thirteenth aspect, the present invention provides a method of purifying a polypeptide of interest comprising:
(a) expressing the hybrid nucleic acid of the third aspect in a cell to produce a hybrid polypeptide comprising the polypeptide of interest and a polymerisable polypeptide;
(b) polymerising the hybrid polypeptide;
(c) purifying the polymerised hybrid polypeptide;
(d) cleaving the polypeptide of interest from the polymerisable polypeptide; and
(e) purifying the polypeptide of interest.

Preferably, the polypeptide of interest is cleaved from the polymerisable polypeptide by a protease which protease is itself linked to a polymerisable polypeptide to form a protease hybrid polypeptide. More preferably, after cleavage, the protease hybrid polypeptide is polymerised and the polypeptide of interest is purified by removal of the polymerised protease hybrid polypeptide.

It will be clear to the skilled addressee that two or more hybrid polypeptides may be used at the same time and that they may comprise different polypeptides of interest or may comprise different polymerisable polypeptides. When the hybrid polypeptides comprise different polymerisable polypeptides the polymerisation requirements for each of the polymerisable polypeptides may differ thus allowing for selective sequestration or purification of the polypeptides of interest linked to the polymerisable polypeptides.

The skilled addressee will recognise that, although one of the benefits of the present invention is that it does not require a matrix for sequestering and/or purifying the polypeptides of interest, the system may also be adapted such that the hybrid polypeptides can be attached to a support. One way in which this could be achieved is by attaching or embedding on a support an agent which can bind the hybrid polypeptide. While it is clear that this agent could bind any part of the hybrid polypeptide, it is preferred that the agent is, itself, a polypeptide which binds the polymerisable polypeptide and, most preferably, the agent is a polymerisable polypeptide which is identical to the polymerisable polypeptide of the hybrid polypeptide, or is a variant thereof.

In the context of the present invention, the term “polypeptide” means a molecule comprising two or more amino acids and includes within its meaning peptides of any length and proteins. The amino acids may be D- or L-amino acids. Further, the amino acids may be modified, for example, by phosphorylation, methylation, glycosylation, acylation or isoprenylation. It will be understood by the skilled addressee that such modification should not entirely abolish the ability of the polymerisable polypeptide to polymerise—although some degree of inhibition of the ability to polymerise may be tolerated. The tolerable degree of inhibition of the ability to polymerise will be readily recognised by the skilled addressee upon simple experimentation—such experimentation being well within the competence of those skilled in the art.

In the context of the present invention, the phrase “polypeptide of interest” is to be construed in the sense of a polypeptide which is to be sequestered and/or purified.

In the context of the present invention, the term “sequestering” includes within its meaning assembling or aggregating a compound, such as the hybrid polypeptide of the present invention, and does not necessarily imply that the compound is removed from its environment. For example, the hybrid polypeptide of the present invention could be “sequestered” within a cell by polymerising it within the cell.

In the context of the present invention, the term “purifying” includes within its meaning separating and/or isolating a compound, such as the hybrid polypeptide of the present invention, from other components and implies no limitation as to the purification method or the degree of purity of a compound which has been purified.

In the context of the present invention, the term “hybrid polypeptide” refers to a polypeptide comprising at least two polypeptides.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”.

It will be clear to the skilled addressee that an analogous meaning should be applied to terms grammatically related to those defined above eg. “purification” should be construed in a manner consistent with the construction of the term “purifying”.

### BRIEF DESCRIPTION OF THE FIGURES

FIG. 1. The nucleotide sequence of the GFP gene from Aequorea Victoria (717 bp) [SEQ ID NO. 1]. The start codon and stop codon are underlined.

FIG. 2. The nucleotide sequence of the FtsZ gene from E. coli (1152 bp) [SEQ ID NO. 2]. The start codon and stop codon are underlined.

FIG. 3. Graphical map of the plasmid pTYB 1. Arrows indicate approximate length of the genes/elements and their orientation. amp-resistance: beta-lactamase gene encoding for ampicillin resistance. M13 ori: origin of replication for the phage M13. ColEI ori: ColEI origin of replication for E. coli. lacI: lac-repressor for IPTG-induced control of T7-promoter.

FIG. 4. Flanking and internal regions of the hybrid polypeptide formed by fusion of FtsZ and GFP. RBS: ribosome binding site.

FIG. 5. Purification and cleavage of a hybrid polypeptide formed by fusion of GFP and FtsZ as analysed by SDS-PAGE (12.5 (w/v) polyacrylamide). Lane M contains a pre-stained broad-range molecular size marker (Bio-Rad) with corresponding sizes on the left. Lane 1 shows that the cleared cell lysate contains a strongly overexpressed band (see upper arrow) of estimated molecular weight of 67 kDa, corresponding well with the combined mass of FtsZ (40.2 kDa) and GFP (27 kDa). Lane 2 shows the supernatant after the first centrifugation step and after addition of MgCl₂, CaCl₂ and GTP. Lane 3 contains a sample of the resuspended pellet after the first centrifugation step and after addition of MgCl₂, CaCl₂ and GTP and shows clearly that the hybrid polypeptide is purified in the pellet. Lane 4 contains a sample of the supernatant after ice-incubation and the second centrifugation and shows clearly that the hybrid polypeptide has been dissociated. Lane 5 contains a sample of the resuspended pellet after the second centrifugation and shows that the hybrid polypeptide is no longer in the pellet. Lane 6 shows the cleavage of the hybrid polypeptide into GFP (lowest arrow) and FtsZ (second lowest arrow) through the action of the Prescission™ protease (46 KDa; see second arrow from top).

FIG. 6. Gene-sequence of the human rhinovirus type 14 genome encoding for the protease 3C [SEQ ID NO. 3].

FIG. 7. Flanking and internal regions of the hybrid polypeptide formed by fusion of E.coli FtsZ and HRP protease 3C (HRP3C). RBS: ribosome binding site.

FIG. 8. Purification of a hybrid polypeptide formed by fusion of HRP protease 3C and FtsZ as analysed by SDS-PAGE (12.5 (w/v) polyacrylamide). Lane M contains a pre-stained broad-range molecular size marker (Bio-Rad) with corresponding sizes on the left. Lane 1 shows that the cleared cell lysate contains a strongly overexpressed band (see arrow) of estimated molecular weight of 60 kDa, corresponding well with the combined mass of FtsZ (40.2 kDa) and HRP protease 3C (20 kDa). Lane 2 shows the supernatant after the first centrifugation step and after addition of MgC l₂, CaCl₂ and GTP. Lane 3 contains a sample of the resuspended pellet after the first centrifugation step and after addition of MgCl₂, CaCl₂ and GTP and shows clearly that the hybrid polypeptide is purified in the pellet. Lane 4 contains a sample of the supernatant after ice-incubation and the second centrifugation and shows clearly that the hybrid polypeptide has been dissociated.

FIG. 9. Cleavage of a hybrid polypeptide (formed by fusion of GFP and FtsZ) by a protease hybrid polypeptide (formed by fusion of HRV protease 3C and FtsZ) and subsequent purification as analysed by SDS-PAGE (15% (w/v) polyacrylamide). Lane M contains a pre-stained broad-range molecular size marker (Bio-Rad) with corresponding sizes on the left. Lane 1 and 2 show the purified hybrid polypeptide and protease hybrid polypeptide (top arrow), respectively. Lane 3 contains a sample of the protease treatment before the addition of MgCl₂, CaCl₂ and GTP. Lane 4 contains a sample of the supernatant after the centrifugation and addition of MgCl₂, CaCl₂ and GTP and shows the purified GFP-protein at around 27 kDa (lower arrow). Lane 5 contains a sample of the resuspended pellet after the centrifugation and addition of MgCl₂, CaCl₂ and GTP and shows that the protease hybrid polypeptide (top arrow) and the cleaved FtsZ protein (middle arrow) have been polymerised and removed.

### DETAILED DESCRIPTION OF THE INVENTION

The present invention relates to a process of sequesting and/or purifying a polypeptide of interest using a polymerisable polypeptide. It has surprisingly been found that when a polypeptide of interest is linked to a polymerisable polypeptide such as a self-polymerising protein, the resultant hybrid polypeptide can be polymerised and, if required, purified.

Recombinant DNA technology can be used to link the polypeptide of interest and the polymerisable polypeptide. For example, DNA encoding a polypeptide of interest can be fused to DNA encoding the polymerisable polypeptide. The fused DNA can be inserted into a cloning vector and an appropriate host transformed. Upon expression, a hybrid polypeptide or fused protein is produced which can be purified by, for example, induced and controlled polymerisation of the hybrid polypeptide and subsequent physical separation based on mass and/or size. The hybrid polypeptide so purified may in certain instances be useful in its hybrid form, or it may be cleaved to provide the polypeptide of interest itself which can be purified alone.

The present invention may be carried out in a manner that removes the requirement for a purification matrix. For example, the hybrid polypeptide can be isolated and purified directly, eg. from a crude cellular extract or culture medium, simply by inducing controlled polymerisation and subsequent physical separation based on mass and/or size (such as centrifugation or filtration).

While the polypeptide of interest (or target protein) may be useful in its hybrid form, it may also be desirable to separate or cleave the polymerisable polypeptide (eg a self-polymerising protein) away from the polypeptide of interest. This may be accomplished in a variety of ways. For example, a DNA fragment coding for a linker polypeptide (or a predetermined peptide) may be employed to link the DNA fragments coding for the polymerisable polypeptide and polypeptide of interest. The linker polypeptide is preferably one which comprises, or consists of, a region recognized and cleaved by a proteolytic agent such that it cuts the hybrid polypeptide at or near the polypeptide of interest—preferably without interfering with the biological activity of the polypeptide of interest. The linker polypeptide, in addition to providing a convenient proteolytic cleavage site, may also serve as a cloning site or polylinker, i.e. by providing multiple DNA restriction sites to facilitate fusion of the DNA fragments coding for the polypeptide of interest and the polymerisable polypeptide, and/or as a spacer which separates the polypeptide of interest and the polymerisable polypeptide and thus, for example, allows access by the proteolytic agent to cleave the hybrid polypeptide. Other linker polypeptides contemplated by the present invention include recognition sites for chemical cleavage (eg. by cyanide bromide) or self-cleavable protein elements like inteins (Xu et al., 2000).

Polymerisable polypeptides which may be employed in accordance with the present invention include self-polymerising proteins such as prokaryotic cell division proteins, like FtsZ, and eukaryotic proteins of the cytoskeleton, like tubulin or actin (Bi & Lutkenhaus, 1991; Bramhill & Thompson, 1994; Erickson et al., 1996; Lallo et al., 1999; Löwe & Amos, 1998; Mukherjee & Lutkenhaus, 1998; Mukherjee & Lutkenhaus, 1999; Yu & Margolin, 1997). The preferred polymerisable polypeptide for practising the present invention is the prokaryotic FtsZ protein or variant thereof. These proteins polymerise under controllable, defined conditions to provide polymeric structures such as fibres, filaments or networks (Löwe & Amos, 1999; Mukherjee &Lutkenhaus, 1999; Yu & Margolin, 1997). Conditions to induce polymerisation include, for example, physical parameters (eg. temperature or concentration) and chemical parameters (eg. pH value, presence of salts or organic compounds).

The so-generated multimeric structures have properties distinct from those of the monomeric form of the proteins—one such property being their greatly increased size and mass. These differences can be utilised to separate the hybrid polypeptide in either its monomeric or multimeric form from other host cell compounds such as other cellular proteins, membranes, nucleic acids or small molecules. Methods of separation include any physical method distinguishing between size and/or mass (eg. centrifugation, filtration, dialysis etc.). If the polymerisation process is reversible (ie. by dissociation of the multimeric structures into monomers) the separation process can be performed repetitively to progressively increase purification levels.

As indicated above, proteolytic agents may be used to cleave the polypeptide of interest from the polymerisable polypeptide. In accordance with the present invention, the proteolytic agent may be fused or linked to the hybrid polypeptide. Preferably it is fused or linked adjacent the polymerisable polypeptide. Hence, the proteolytic agent can be used to cleave the polypeptide of interest from the polymerisable polypeptide and the proteolytic agent can subsequently be removed by polymerising the polymerisable polypeptide (with the proteolytic agent attached) followed by physical separation of the polymerised product from the polypeptide of interest using techniques mentioned above.

Preferred embodiments of the invention will now be described, by way of example only, with reference to the accompanying Figures. An overview of the experimental procedure is provided followed by the actual experimental data.

I. Preparation of Vector

(a) The DNA (deoxyribonucleic acid) encoding for the desired polymerisable polypeptide eg. a self-polymerising protein, is purified.
(b) The DNA is inserted into a suitable cloning vector with a selectable marker and the mixture is used to transform an appropriate host such as E. coli.
(c) The transformants are selected based on, for example, their resistance to antibiotics or other phenotypic characteristic conferred by the selectable marker.
(d) The plasmid DNA is prepared from the selected transformants.
(e) The polymerising properties of the polypeptide are determined.
(f) The flanking regions of the gene encoding the polymerisable polypeptide can be manipulated using standard genetic techniques to generate suitable multiple cloning sites, ie. DNA regions encoding recognition sites of proteolytic agents or other elements of similar function.
   II. Insertion of DNA Coding for the Polypeptide of Interest into the Vector
(a) The DNA encoding the polypeptide of interest (or target protein) is purified.
(b) This DNA fragment is inserted into the vector described in I above by standard genetic techniques so that an in-frame fusion is formed between the DNA fragment coding for the polypeptide of interest and for the DNA fragment coding for the polymerisable polypeptide.
(c) The vector containing this hybrid nucleic acid is introduced into an appropriate host.
   III. Expression and Purification of the Hybrid Polypeptide
(a) The host cell containing the hybrid nucleic acid described in II above is cultured.
(b) Expression of the hybrid polypeptide is induced by conventional methods.
(c) A cell extract containing the expressed hybrid polypeptide is prepared by standard techniques.
(d) Insoluble compounds may be removed from the cell extract by centrifugation or comparable techniques.
(e) Polymerisation of the hybrid polypeptide is induced in the (cleared) cell extract through defined chemical and/or physical conditions.
(f) The polymerised hybrid polypeptide is separated from other constituents of the cell extract by physical methods differentiating on the basis of size and/or mass (eg. centrifugation).
(g) Dissociation (depolymerisation) of the hybrid polypeptide is induced through defined chemical and/or physical conditions.
(h) The dissociated hybrid polypeptide is separated from other contaminating compounds by physical methods differentiating on the basis of size and/or mass (eg. centrifugation).

Steps (e) to (h) may be repeated until a suitable purification level of the hybrid polypeptide is reached. For some applications, a first polymerisation without further dissociation/polymerisation will be sufficient.

IV. Cleavage of the Hybrid Polypeptide and Separation of the Polypeptide of Interest

(a) The polypeptide of interest is released from the hybrid polypeptide by addition of a proteolytic agent to the purified hybrid polypeptide. The hybrid polypeptide is preferably in its dissociated form when the proteolytic agent is active. Preferably the proteolytic agent itself is fused or linked to the polymerising polypeptide. Linkage may be by a linker polypeptide.
(b) Re-polymerisation of the dissociated polymerisable polypeptide and the proteolytic agent fused or linked to the polymerisable polypeptide is induced through defined chemical and/or physical conditions.
(c) The polymerised polymerisable polypeptide fused to the proteolytic agent may be separated from the polypeptide of interest by physical methods differentiating on the basis of size and/or mass (eg. centrifugation).
   Polymerisable Polypeptide

The polymerisable polypeptide may be a self-polymerising protein, for example, a prokaryotic cell division protein like FtsZ or a eukaryotic protein of the cytoskeleton like tubulin or actin. The preferred protein for practising the present invention is the prokaryotic FtsZ protein or variation thereof.

The product of the prokaryotic geneftsz of E. coli, ie the protein FtsZ, is part of the cell division ring, which occurs during the formation of two daughter cells during cell division (Bi & Lutkenhaus, 1991). The FtsZ protein is a GTPase (GTP hydrolysing enzyme) that is essential for cell division in E. coli. FtsZ in its monomeric form is 40.3 kDa large and approximately 4-5 nm in diameter. FtsZ has been shown to polymerise in vitro into long filaments upon hydrolysis of GTP (Bramhill & Thompson, 1994). These filaments can be so-called protofilaments with a diameter of 4-5 nm or protofilament bundles and networks with sizes greater than 1 mm and weights of several thousands kDa (Yu & Margolin, 1997). These large, multimeric structures can be separated from the monomeric FtsZ by centrifugation. The FtsZ-filaments dissociate upon depletion or removal of GTP into the monomeric form (Bramhill & Thompson, 1994). In addition, FtsZ-filaments can be stabilised by divalent cations such as calcium even after GTP has been depleted. Further, dissociation can be achieved again through subsequent removal of the divalent cation (Yu & Margolin, 1997). FtsZ has been also been converted into an ATPase (ATP hydrolysing enzyme) through protein engineering techniques and opens therefore the possibility to replace GTP by ATP in the present invention (RayChaudhuri & Park, 1994).

The present invention also contemplates the use of other mutants of FtsZ with altered polymerisation and/or GTPase properties. For example, replacement of the aspartate residue with a cysteine or asparagine residue at position 212 of E. Coli FtsZ allows for a solely cation-induced polymerisation (ie without the addition of GTP). The polymerisation of this mutant protein can be induced by magnesium, calcium, nickel, cobalt, zinc or manganese and is reversible after the removal of the cation (Scheffer et al., 2001). Other mutations in FtsZ at positions 70 (alanine to threonine), 209 (aspartate to alanine) or 269 (aspartate to alanine) also showed reduced GTPase activity but retained wild-type polymerisation properties (Lu et al., 2001).

Linker Polypeptide

A DNA fragment coding for a linker polypeptide (or predetermined peptide) may be employed to link the DNA fragments coding for the polymerisable polypeptide and the polypeptide of interest. The linker polypeptide is preferably one which is recognized and cleaved by a proteolytic agent such that it cuts the hybrid polypeptide without interfering with the biological or chemical activity of the polypeptide of interest and the polymerisable polypeptide. One such linker polypeptide is described in Cordingley et al. (Cordingley et al., 1990). The amino acid sequence of the recognition site is Leu-Glu-Val-Leu-Phe-Gln-Gly-Pro and it is cleaved between the glutamine and the glycine residues by the 3C-protease from human rhinovirus type 14 (HRV protease 3C). Other examples of proteases (and their corresponding recognition sites) are thrombin (Leu-Val-Pro-Arg-Gly-Ser), Factor Xa (Ile-Glu-Gly-Arg) and enterokinase (Asp-Asp-Asp-Asp-Lys).

As noted above, the linker polypeptide in addition to providing a convenient proteolytic cleavage site, may also serve as a (multiple) cloning site, ie. by providing multiple restriction sites to facilitate fusion of the DNA fragments coding for the polypeptide of interest and the polymerisable polypeptide, and/or as a spacer which separates the polypeptide of interest and the polymerisable polypeptide. The spacer may allow access by the proteolytic agent to cleave the hybrid polypeptide or avoid unfavourable steric interference between the polypeptide of interest and the polymerisable polypeptide.

Other linker polypeptides contemplated by the present invention comprise recognition sites for chemical cleavage (eg. by cyanide bromide) or self-cleavable protein elements like inteins (Xu et al., 2000).

Polypeptide of Interest

The present invention can be applied to any polypeptide of interest. Such polypeptides may be proteins including enzymes such as endonucleases, methylases, oxidoreductases, transferases, hydrolases, lysases, isomerases or ligases.

The present invention also contemplates the use of the invention in respect of proteins, such as ferritin or ovalbumin or transport proteins, such as hemoglobin, serum albumin or ceruloplasmin.

The present invention also contemplates the use of the invention in respect of antigens or antigenic determinants which can be used in the preparation of vaccines or diagnostic reagents.

The present invention also contemplates the use of the invention in respect of proteins that serve a protective or defence function, such as the blood protein thrombin and fibrinogen. Other protective proteins which may be used include the binding proteins, such as antibodies or immunoglobulins that bind to and thus neutralise antigens.

The proteins to which the present invention may be applied may also encompass various hormones such as Human Growth Hormone, somatostatin, prolactin, estrone, progesterone, melanocyte, thyrotropin, calcitonin, gonadotropin and insulin. Other such hormones include those that have been identified as being involved in the immune system, such as interleukin 1, interleukin 2, colony stimulating factor, macrophage-activating factor and interferon.

Proteins that serve as structural elements may also be used in the present invention. Such proteins include, for example, the fibrous proteins collagen, elastin and alpha-keratin. Other structural proteins which may be used in the present invention include, for example, glyco-protein, virus-protein and muco-protein.

It is also contemplated that the method of the invention may be applied to a number of polypeptides of interest (eg. open reading frames of an organism) whereby the polypeptides may be purified simultaneously in a multichannel apparatus such as a microtitre plate coupled with a microtitre plate centrifuge or a manifold filter apparatus.

In addition to the above-noted naturally occurring proteins, the present invention may be employed to sequester and/or purify synthetic polypeptides or proteins defined generally as any sequence of amino acids not occurring in nature.

Preparation of Hybrid Nucleic Acid and Expression Vectors

Various procedures and materials for preparing recombinant vectors, transforming host cells with the vectors, replicating the vectors and expressing polypeptides and proteins will be known to the skilled addressee—some of which are discussed by Sambrook et al. (Sambrook et al., 1989).

In practising the present invention, various cloning vectors may be utilised. Although the preferred vector is a plasmid, the skilled addressee will appreciate that the vector may also be, for example, a phage or a virus. If a plasmid is employed, it may be obtained, for example, from a natural source or may be artificially synthesised. The plasmid chosen is preferably compatible with the particular cells serving as the host, whether they be, for example, bacterial (such as E. coli), fungal (yeast) or others.

The plasmid may also have a suitable origin of replication (replicon) for the particular host cell chosen.

The plasmid cloning vector preferably includes restriction enzyme sites to allow for cleavage of the plasmid for subsequent ligation with the foreign genes without causing inactivation of the replicon and also providing suitable ligatable termini that are complementary to the termini of the foreign genes being inserted. To this end, it would be useful for the plasmid to have single substrate sites for a large number of restriction endonucleases.

Moreover, the plasmid should preferably have a phenotypic property that will enable the transformed host cells to be readily identified and separated from cells which do not undergo transformation. Such phenotypic selection genes may include genes providing resistance to a growth inhibiting substance, such as an antibiotic. Plasmids are now widely available that include genes resistant to various antibiotics, such as tetracycline, streptomycin, sulfa-drugs, and ampicillin. When host cells are grown in a medium containing one of these antibiotics, only transformants having the appropriate resistant gene will survive.

To prepare the chosen plasmid for ligation, it is preferably digested with a restriction endonuclease to produce a linear segment(s) in which the two DNA strands are cleaved at closely adjacent sites to produce cohesive termini (“sticky ends”) bearing 5′-phosphate- and 3′-hydroxyl groups, thereby facilitating ligation with the foreign genes. For the plasmids identified above, restriction endonucleases will produce this result.

Certain restriction enzymes (PvuII, SmaI) may result in the formation of blunt ends. The blunt ends of the plasmid can be joined to the foreign genes with T4 DNA ligase. The methods and materials for achieving efficient cleavage and ligation are well known in the art.

Prior to being joined with the selected cloning vector, it is desirable that the foreign genes coding for the polymerisable polypeptide and the polypeptide of interest be first joined together to form a hybrid nucleic acid. Ideally, the nucleic acids encoding the polypeptide of interest and the polymerisable polypeptide are treated with the same restriction endonuclease used to cleave the plasmid vector so that the termini will be compatible with the corresponding termini of the plasmid. The nucleic acids may also be treated with a second, different restriction endonuclease to prepare the appropriate ends of the nucleic acids for ligation to each other.

The nucleic acids may then be ligated to the linearized plasmid fragment in a solution with DNA ligase. After incubation, the recircularized plasmid having the correct orientation of the cointegrated genes can be identified by standard techniques such as, for example, gel electrophoresis.

Transformation of Recombinant Plasmid

The recombinant DNA plasmids, as prepared above, may be used for the transformation of host cells. Although the host cell may be any appropriate prokaryotic or eukaryotic cell, preferably it is a well-defined bacterium, such as E. coli or a yeast strain. Both such hosts are readily transformed and capable of rapid growth in fermentation cultures. Cells from other organisms can be employed, for instance fungi and algae. In addition, other forms of bacteria such as Salmonella or Pneumococcus may also be useful. The host is preferably one that has the necessary biochemical pathways for phenotypic expression and other functions for proper expression of the hybrid polypeptide. The techniques for transforming recombinant plasmids into E. coli strains are widely known. A typical protocol is set forth in Sambrook et al. (Sambrook et al., 1989).

In transformation protocols, generally only a portion of the host cells is actually transformed, due to limited plasmid uptake by the cells. Thus, before transformants are isolated, the host cells used in the transformation protocol typically are multiplied in an appropriate medium. The cells that actually have been transformed can be identified by placing the original culture on agar plates containing a suitable growth medium containing the phenotypic identifier, such as an antibiotic. Only those cells that have the correct antibiotic resistance gene will survive. Cells from the colonies that survive can be lysed and then the plasmid isolated from the lysate. The plasmid thus isolated can be characterised, eg. by digestion with restriction endonucleases and subsequent gel electrophoresis or by other standard methods.

Once transformed cells are identified, they can be multiplied by established techniques, such as by fermentation. In addition, the recovered cloned recombinant plasmids can be used to transform other strains of bacteria or other types of host cells for large-scale replication and expression of the hybrid polypeptide comprising the polypeptide of interest.

Purification of the Hybrid Polypeptide

The hybrid polypeptide may be released from the host cell by appropriate lysis techniques that are able to destroy the cell membrane or cell wall structure of the host cell. These include, but are not restricted to, physical methods such as freeze-thawing, french press rupturing, bead beating, sonication, or chemical-enzymatic systems such as lysozyme-treatment or detergent-lysis. The skilled addressee will be aware of many such techniques—some of which are discussed by Deutscher (Deutscher, 1990).

Polymerisation of the hybrid polypeptide may be induced in the host cell lysate cell extract through defined chemical and/or physical conditions. For example, for a hybrid polypeptide comprising E. coli FtsZ as the polymerisable polypeptide this can be done through the addition of GTP, magnesium chloride and calcium chloride and incubation at 37° C.

Filaments, bundles or networks containing polymerised hybrid polypeptide may be separated from smaller compounds of the cell lysate by physical techniques discriminating on the basis of size and/or weight. Preferably a method and its parameters are chosen in such a way that the size and/or weight cut-off is close to the size and/or weight of the filaments, bundles or networks produced under the given conditions of polymerisation. The skilled addressee will note that the technique of centrifugation provides a wide range of size and/or weight cut-offs through variation of rotational force and/or time. For example, filaments, bundles or networks containing polymerised hybrid polypeptide comprising E. coli FtsZ can be readily recovered by 20 min centrifugation at 20 000× g. Other preferred methods of separation include differential sedimentation, filtration, dialysis and flow sorting.

The polymerisation products can be subsequently dissociated through changes in the defined chemical and/or physical conditions. For example, for polymerised hybrid polypeptides comprising E. coli FtsZ as the polymerisable polypeptide this can be done through the removal of GTP and calcium chloride and incubation at 4° C. Methods to remove or diminish chemical substances include, for example, dialysis, dilution, absorption, enzymatic or chemical degradation used alone or in combination and are known to the skilled addressee.

The dissociated hybrid polypeptide may be separated from larger compounds by physical techniques discriminating on the basis of size and/or weight and techniques suitable are mentioned above.

It is a preferred feature of the present invention that the polymerisation and dissociation of the hybrid polypeptide be repeated in an alternating fashion so that substances larger and smaller than the hybrid polypeptide are removed. This alternating/cycling between the two physical states of the hybrid polypeptide (large, assembled state and small, dissociated state) can thus be repeated until desired purification levels are achieved.

While repetitive cycling as described above may provide high purity, sufficient purification levels might already be achieved after one initial polymerisation. It is therefore also contemplated that cleavage and release of the polypeptide of interest can take place directly after this stage. As such, polymerisable polypeptides encompassed in the present invention also include those which polymerise in an irreversible way or those for which polymerisation conditions can be set such that dissociation is not possible.

Separation of the Polypeptide of Interest from the Hybrid Polypeptide

The hybrid polypeptide purified as described above may be cleaved, for example, by sequence specific proteases such as a HRV protease 3C or by discrete chemical cleavage, such as cyanogen bromide. The proteolytic agent or chemical cleavage agent and the polymerisable polypeptide cleaved from the hybrid polypeptide can be separated from the polypeptide of interest by suitable protein purification methods such as described by Deutscher (Deutscher 1990).

Alternatively, the proteolytic agent itself may be fused to a polymerisable polypeptide which may be the same as the polymerisable polypeptide fused to the polypeptide of interest. This protease hybrid polypeptide can be produced and purified in the same manner as described for the hybrid polypeptide mentioned above. The protease hybrid polypeptide can be used to cleave the hybrid polypeptide between the polymerisable polypeptide and the polypeptide of interest releasing the two polypeptides. The protease hybrid polypeptide and the released polymerisable polypeptide can be separated from the released polypeptide of interest using the polymerising properties of the polymerisable polypeptide(s) and physical separation technique as described above.

The Examples below describe specific exemplary protocols to illustrate the invention.

### EXAMPLE 1

This example describes a method of cloning, expressing and purifying a hybrid polypeptide (formed by fusion of Aequorea victoria green fluorescent protein (GFP) and E. coli FtsZ) and its proteolytic cleavage. The GFP is a representative “polypeptide of interest” and the FtsZ is a polymerisable polypeptide.

(a) Preparation of the Vector

The gene encoding the green fluorescent protein (GFP) from Aequorea victoria was fused with the gene encoding the FtsZ protein from E. coli. This chimeric construct (hybrid nucleic acid) was then cloned into a modified vector based on the vector pTYB 1 (New England Biolabs, Beverly, Mass., USA). The protocols used were as follows:

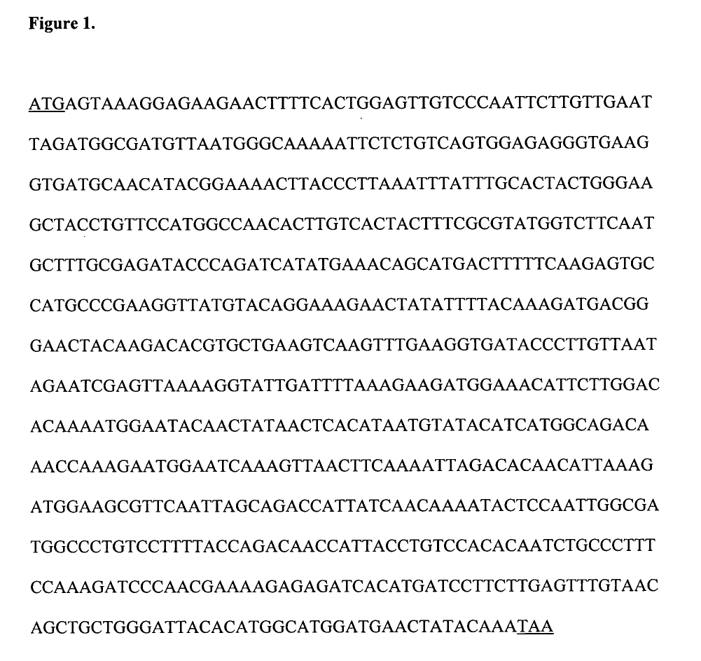

The GFP gene was amplified from a mini-TN10 transposon containing a promoterless GFP gene by polymerase chain reaction (PCR) with primers incorporating endonuclease restriction sites immediately upstream and downstream of the gene. The full sequence of the GFP gene can be found in FIG. 1. The PCR-conditions were as follows: 1 ng of mini-Tn10 transposon DNA was used as a template. The PCR buffer contained 10 mM tris(hydroxymethyl)aminomethyl hydrochloride (Tris-HCl) (pH 9 at 25° C.), 50 mM potassium chloride (KCl) and 0.1% (v/v) Triton X-100, 2.5 mM magnesium chloride (MgCl₂), 0.4 mM of dNTP, 1 unit Taq DNA polymerase (Promega, Madison, Wis., USA) and 0.1 unit of Pfu DNA polymerase (Promegal in a 20 microliter reaction volume. The PCR primers were GFPBsp (5′ ATCATGAGTAAAGGAGAAGAACTTTTC 3′) [SEQ ID NO. 4] incorporating a BspH] site (underlined) and GFPBam (5′ AGGATCCTTATTTGTATAGTTCATCCATG 3′) [SEQ ID NO. 5] incorporating a BamHI site (underlined) and were added in a concentration of 0.5 micromolar. The PCR reaction mixture (without the polymerase) was heated to 95° C. for 1 min and then at 80° C. for 1 min at which point the DNA polymerase mix was added. The mixture was then heated for 25 cycles at 95° C. for 15 sec, 50° C. for 20 sec and 72° C. for 1 min. A final 7 min cycle at 72° C. followed this. One microlitre of the reaction mix was cloned into the vector pCR®-BluntII-TOPO® using the Zero Blunt® TOPO® PCR cloning kit (Invitrogen, Carlsbad, Calif., USA) according to the manufacturer's instructions. Recombinant plasmids containing the GFP gene were purified using the Quantum Prep® Plasmid Miniprep Kit (Bio-Rad, Hercules, Calif., USA) according to the manufacturer's instructions and analysed by restriction digest with the restriction endonucleases BamHI and BspHI (New England Biolabs, Beverly, Mass., USA) according to the manufacturer's recommendations. Digested plasmid DNA was separated on a 1% (w/v) agarose gel with TAE-buffer (40 mM Tris-acetate, 1 mM ethylenediaminetetraacetic acid (EDTA)), stained with ethidium bromide and visualised under UV irradiation. Three bands were visible and the smallest one (approximately 700 bp in size) was cut out and placed into a centrifuge tube. The DNA was eluted from the gel slice by addition of 0.5 M sodium chloride (NaCl) solution and subsequent incubation for 30 min at room temperature. The gel slice was placed in a standard spin column (Bio-Rad), frozen for 10 min at −20° C. and then spun for 13 min at room temperature and 15 000× g. The flowthrough was recovered and the DNA precipitated using standard techniques described in Sambrook et al. (Sambrook et al., 1989). The final DNA pellet was resuspended in 20 microlitres of water.

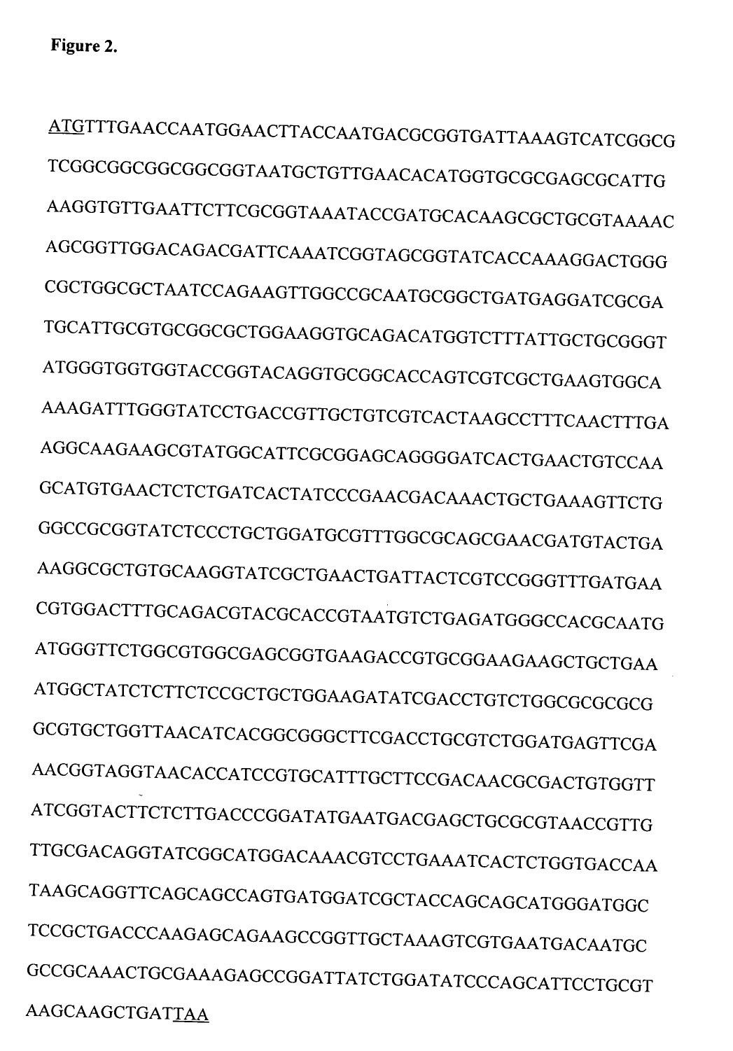

The FtsZ gene was amplified from E. coli genomic DNA by PCR with primers incorporating endonuclease restriction sites and sequences encoding linker polypeptides immediately upstream and downstream of the gene. The full sequence of the FtsZ gene can be found in FIG. 2. The PCR-conditions were as follows: 20 ng of E. coli genomic DNA was used as a template. The PCR-buffer contained 10 mM Tris-HCl (pH 9 at 25° C.), 50 mM KCl and 0.1% (v/v) Triton® X-100, 2.5 mM MgCl₂, 0.4 mM of dNTP, 1 unit Taq DNA polymerase (Promega) and 0.1 unit of Pfu-polymerase (Promega) in a 20 microlitre reaction volume. The PCR-primers were FFNde (5′ GGCATATGTTTGAACCAATGGAAC 3′) [SEQ ID No. 6] incorporating a NdeI site (underlined) and FRNco (5′ GTCCATGGGCCCTTGAAATAGTACTTC 3′) [SEQ ID NO. 7] incorporating a NcoI site (underlined) and were added at a concentration of 0.5 micromolar. An additional primer FRL (5′ GGGCCCTTGAAATAGTACTTCTAGATCAGCTTGCTTACGCAGG 3′) [SEQ ID NO. 8] was added at a concentration of 2.5 picomolar. The PCR reaction mixture (without the DNA polymerase) was heated to 95° C. for 1 min and then at 80° C. for 1 min at which point the DNA polymerase mix was added. The mixture was then heated for 30 cycles at 95° C. for 15 sec, 55° C. for 20 sec and 72° C. for 2 min. A final 7 min cycle at 72° C. followed this. One microliter of the reaction mix was cloned into the vector pCR®-BluntII-TOPO® using the Zero Blunt® TOPO® PCR cloning kit (Invitrogen) according to the manufacturer's instructions. Recombinant plasmids containing the FtsZ gene were purified using the Quantum Prep® Plasmid Miniprep Kit (Bio-Rad) according to the manufacturer's instructions and analysed by restriction digest with the restriction endonucleases NdeI and NcoI (New England Biolabs) according to the manufacturer's recommendations. Digested plasmid DNA was separated on a 1% (w/v) agarose gel with TAE-buffer, stained with ethidium bromide and visualised under UV irradiation. Four bands were visible and the second smallest one (approximately 1200 bp in size) was cut out and placed into a centrifuge tube. The DNA was eluted from the gel slice by addition of 0.5 M NaCl solution and subsequent incubation for 30 min at room temperatures. The gel slice was placed into a standard spin column (Bio-Rad), frozen for 10 min at −20° C. and then spun for 13 min at room temperature and 15 000× g. The flowthrough was recovered and the DNA precipitated using standard techniques as described previously (Sambrook et al., 1989). The final DNA pellet was resuspended in 20 microlitres of water.

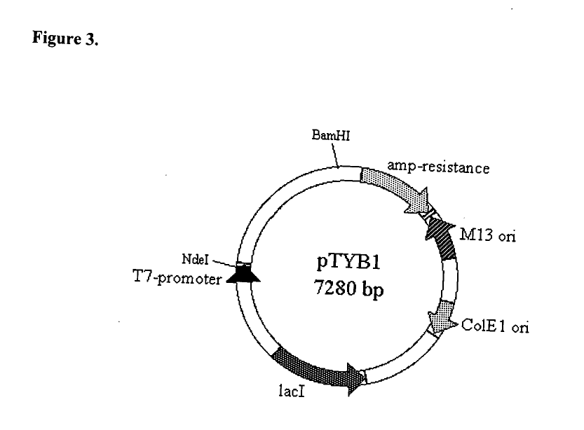

The vector backbone was generated by digesting the plasmid pTYB1 (New England Biolabs) with the restriction enzymes NdeI and Bam HI (both New England Biolabs) according to the manufacturer's recommendations. This releases both a 1.4 kb fragment and a 5.8 kb fragment—the latter which contains all the necessary elements for selection, replication, induction and expression. A plasmid map is shown in FIG. 3.

Digested plasmid DNA was separated on a 1% (w/v) agarose gel with TAE-buffer, stained with ethidium bromide and visualised under UV irradiation. Two bands were visible and the larger one (approximately 5.8 kb in size) was cut out and the agarose slice placed into a centrifuge tube. The DNA was eluted from the gel slice by addition of 0.5 M sodium chloride (NaCl) solution and subsequent incubation for 30 min at room temperatures. The gel slice was placed into a standard spin column (Bio-Rad), frozen for 10 min at −20° C. and then spun for 13 min at room temperature and 15 000× g. The flowthrough was recovered and the DNA precipitated using techniques as described previously (Sambrook et al., 1989). The final DNA pellet was resuspended in 20 microlitres of water. The vector DNA was subsequently dephosphorylated using Shrimp Alkaline Phosphatase (Roche, Basel, Switzerland) according to the manufacturer's recommendations.

Ligation of the digested and purified GFP DNA fragment, FtsZ DNA fragment and pTYB1 DNA fragment was performed as follows. Fifteen nanograms of dephosphorylated pTYB1 fragment was ligated with 5 ng of the GFP DNA fragment and 5 ng of FtsZ DNA fragment in a 10 microlitre volume using T4 DNA ligase and ligation buffer (New England Biolabs) according to the manufacturer's recommendations. Ligation was performed overnight at 14° C. and the reaction stopped by a 10 min incubation at 65° C. Five microlitres of the ligation mixture was transformed into chemical-competent E. coli XL10-Gold® cells (Stratagene, La Jolla, Calif., USA) according to the manufacturer's recommendations. Recombinant plasmids containing the GFP gene fused to the FtsZ gene were purified using the Quantum Prep® Plasmid Miniprep Kit (Bio-Rad) according to the manufacturer's instructions and checked by restriction digestion with the restriction endonucleases NdeI and BamHI (New England Biolabs) according to the manufacturer's recommendations. Digested plasmid DNA was separated on a 1% (w/v) agarose gel with TAE-buffer, stained with ethidium bromide and visualised under UV irradiation. Two bands with sizes of 5.8 and 1.9 kb were detected corresponding to the vector backbone and the fusion between the FtsZ gene and GFP gene, respectively. The entire DNA sequence of the insert DNA was determined by DNA sequencing. The resulting vector was termed pZTAGGFP.

(b) Expression and Purification of the Hybrid Polypeptide (FtsZ fused to GFP)

The plasmid pZTAGGFP was transformed into E. coli strain ER2556 (genotype: F⁻λ⁻fhuA2 [lon] ompT lacZ::T7 geneI gal sulA11 Δ(mcrC-mrr)11::IS10 R(mcr-73::miniTn10)2 R(zgb-210::Tn10) 1 (TetS) endA1 [dcm]; New England Biolabs) prepared and transformed by standard techniques as described in Sambrook et al. (Sambrook et al., 1989). Transformed cells were selected on LB agar plates (10 g/L NaCl, 10 g/L tryptone, 5 g/L yeast extract, 15 g/L agar) containing 100 mg/L ampicillin. After overnight incubation at 37° C. a single ampicillin-resistant colony from the transformation plate was used to inoculate 2 mL of liquid LB-medium (10 g/L NaCl, 10 g/L tryptone, 5 g/L yeast extract) containing 100 mg/L ampicillin. After overnight incubation at 37° C. 200 microlitres of this culture was used to inoculate a 20 mL culture of liquid LB-medium containing 100 mg/L ampicillin. At an optical density at 610 nm (OD₆₁₀) of 0.4-0.5 isopropyl β-D-thiogalactopyranoside (IPTG) was added to the culture to give a final concentration of 1 millimolar. The culture was shifted from 37° C. to 14° C. and incubated for 16 h.

After incubation the cells were harvested by centrifugation at 4° C. for 2 min at 3500× g. The culture supernatant was removed and the cell pellet resuspended in 1 mL of 50 mM Tris-HCl (pH 7.5), 10% (v/v) glycerol and 1 mM phenylmethylsulfonyl fluoride (PMSF). The cells were lysed by 4×15 sec sonication using a Branson Microtip Sonicator at duty setting 80% and output level 2. The cell lysate was cleared from intact cells and large, insoluble material (such as cell membranes) by 20 min centrifugation at 4° C. and 20000× g. The pellet was discarded and the supernatant was adjusted to 1 mM MgCl₂, 20 mM calcium chloride (CaCl₂) and 1 mM GTP. The supernatant was incubated for 30 min at 37° C. and then centrifuged for 20 min at 4° C. and 20,000× g. The supernatant was discarded and the pellet containing the polymerised hybrid polypeptide was resuspended in 1 mL of 50 mM Tris-HCl (pH 7.5) and 10% (v/v) glycerol. The suspension was incubated for 30 min on ice and then centrifuged for 20 min at 4° C. and 20,000× g. The supernatant containing the dissociated hybrid polypeptide was recovered and the pellet resuspended in 1 mL water.

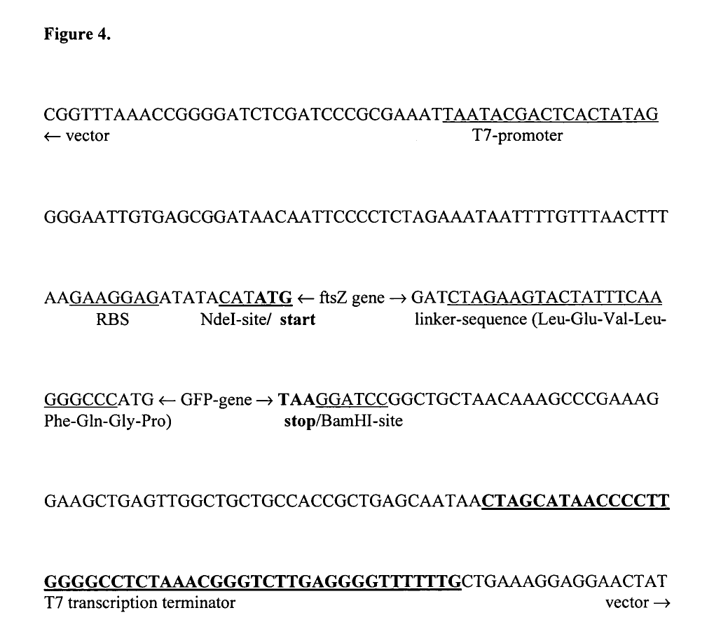

To release the polypeptide of interest from the hybrid polypeptide, 50 microlitres of the purified protein was incubated with 2 units of Prescission™ protease (Amersham Pharmacia, Piscataway, N.J., USA) and the addition of 1 mM dithiothreitol (DTT) for 16 h at 15° C. The Prescission protease™ is a recombinant human rhinovirus protease 3C and cleaves the amino acid sequence Leu-Glu-Val-Leu-Phe-Gln-Gly-Pro after the glutamine residue. This amino acid sequence is part of the linker polypeptide between the FtsZ and the GFP protein produced in the above manner (see FIG. 4).

During the purification procedure samples were taken at all stages and analysed by discontinuous sodium dodecyl sulfate -polyacrylamide gel electrophoresis (SDS-PAGE) as described by Laemmli, (Laemmli, 1970) and Deutscher (Deutscher, 1990). The gel was stained with Coomassie Brilliant blue R250 using standard protocol as described by Ausubel et al. (Ausubel et al., 1994).

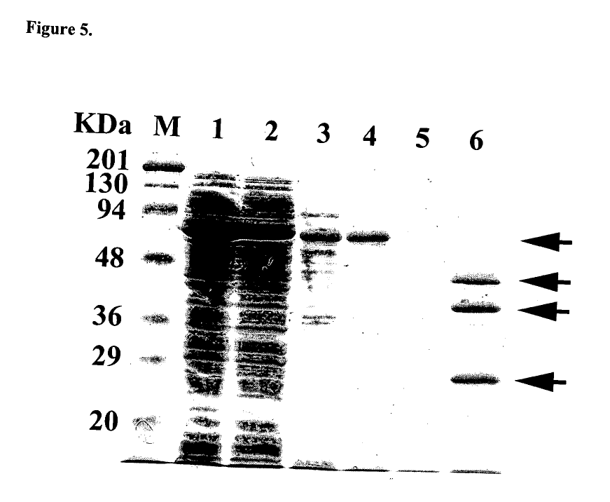

FIG. 5 shows the extent of purification. Lane 1 shows that the cleared cell lysate contains a strongly overexpressed band (see arrow) of estimated molecular weight of 67 kDa, corresponding well with the combined mass of FtsZ (40.2 kDa) and GFP (27 kDa). Lane 2 shows the supernatant after the first centrifugation step and after addition of MgCl₂, CaCl₂ and GTP. Lane 3 contains a sample of the resuspended pellet after the first centrifugation step and after addition of MgCl₂, CaCl₂ and GTP and shows clearly that the hybrid polypeptide is purified in the pellet. Lane 4 contains a sample of the supernatant after ice-incubation and the second centrifugation and shows clearly that the hybrid polypeptide has been dissociated. Lane 5 contains a sample of the resuspended pellet after the second centrifugation and shows that the hybrid polypeptide is no longer in the pellet. Lane 6 shows the cleavage of the hybrid polypeptide through cleavage with Prescission™ protease.

### EXAMPLE 2

This example describes the cloning, expressing and purifying of a hybrid polypeptide (formed by fusion of human rhinovirus (HRV) protease 3C and E. coli FtsZ).

(a) Preparation of the Vector

The gene encoding the HRV protease 3C was fused with the gene encoding for FtsZ from E. coli. One difference between this and the previous example (Example 1) is that a non-cleavable linker polypeptide is employed. This chimeric construct is then cloned into a modified vector based on the vector pTYB1 (New England Biolabs).

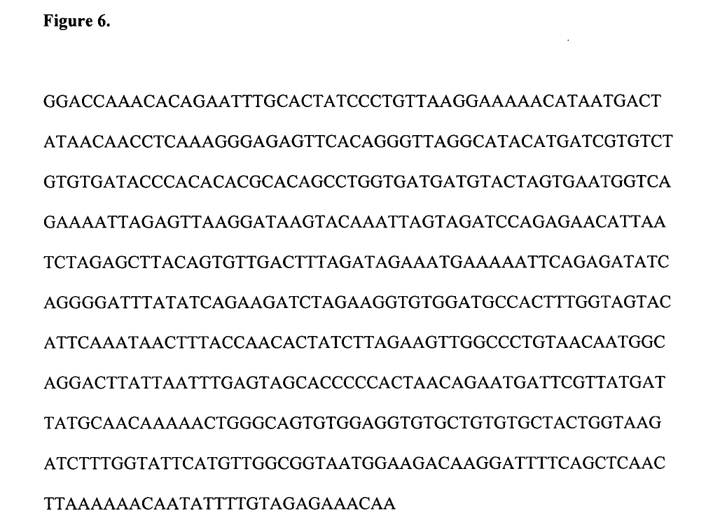

The HRV protease 3C gene was amplified from the plasmid pLJ111 containing the gene by PCR (Leong et al., 1992). Primers were used to incorporate endonuclease restriction-sites immediately upstream and downstream of the gene. The full sequence of the HRV protease 3C gene can be found in FIG. 6. The PCR-conditions were as follows: 1 pg of plasmid pLJ111 was used as a template. The PCR buffer contained 10 mM Tris-HCl (pH 9 at 25° C.), 50 mM KCl and 0.1% (v/v) Triton e X-100, 2.5 mM Mgl₂, 0.4 mM dNTP, 1 unit Taq DNA polymerase (Promega) and 0.1 unit of Pfu DNA polymerase (Promega) in a 20 microlitre reaction volume. The PCR-primers were 3CPNCO (5′ CGCCATGGGACCAAACACAGAATTTGC 3′) [SEQ ID NO. 9] incorporating a NcoI site (underlined) and 3CPBAM (5′ GCGGATCCCTATTGTTTCTCTACAAAATATTG 3′) [SEQ ID NO. 10] incorporating a BamHI site (underlined) and were added to a final concentration of 0.5 micromolar. The PCR reaction mixture (without the DNA polymerase mix) was heated to 95° C. for 1 min and then at 80° C. for 1 min at which point the DNA polymerase mix was added. The mixture was then heated for 35 cycles at 95° C. for 15 sec, 48° C. for 10 sec and 72° C. for 1 min. A final 7 min cycle at 72° C. followed this. One microlitre of the reaction mix was cloned into the vector pCR®-BluntII-TOPO® using the Zero Blunt® TOPO® PCR cloning kit (Invitrogen) according to the manufacturer's instructions. Recombinant plasmids containing the HRV protease 3C gene were purified using the Quantum Prep® Plasmid Miniprep Kit (Bio-Rad) according to the manufacturer's instructions and analysed by restriction digest with the restriction endonuclease BamHI and NcoI (New England Biolabs) according to the manufacturer's recommendations. Digested plasmid DNA was separated on a 1% (w/v) agarose gel with TAE-buffer, stained with ethidium bromide and visualised under UV irradiation. Four bands were visible and the second smallest one (approximately 500 bp in size) was cut out and placed into a centrifuge tube. The DNA was eluted from the gel slice by addition of 0.5 M NaCl solution and subsequent incubation for 30 min at room temperature. The gel slice was placed into a standard spin column (Bio-Rad), frozen for 10 min at −20° C. and then spun for 13 min at room temperature and 15 000× g. The flowthrough was recovered and the DNA precipitated using standard techniques as described previously (Sambrook et al., 1989). The final DNA pellet was resuspended in 20 microlitres of water.

The FtsZ gene was amplified from E. coli genomic DNA by PCR with primers incorporating endonuclease restriction sites and a linker sequence immediately upstream and downstream of the gene. The full sequence of the FtsZ gene can be found in FIG. 2. The PCR conditions were as follows: 20 ng of E. coli genomic DNA were used as a template. The PCR-buffer contained 10 mM Tris-HCl (pH 9 at 25° C.), 50 mM KCl and 0.1% (v/v) Triton® X-100, 2.5 mM MgCl₂, 0.4 mM dNTP, 1 unit Taq DNA polymerase (Promega) and 0.1 unit of Pfu DNA polymerase (Promega) in a 20 microlitre reaction volume. The PCR-primers were FFNde (5′ GGCATATGTTTGAACCAATGGAAC 3′) [SEQ ID NO. 11] incorporating a NdeI site (underlined) and FTSNco (5′ CGCCATGGCAGCTTGCTTACGCAGG 3′) [SEQ ID NO. 12] incorporating a NcoI site (underlined) and were added to a final concentration of 0.5 micromolar. The PCR reaction mixture (without the DNA polymerase mix) was heated to 95° C. for 1 min and then at 80° C. for 1 min at which point the DNA polymerase mix was added. The mixture was then heated for 30 cycles at 95° C. for 15 sec, 55° C. for 20 sec and 72° C. for 2 min followed by a final 7 min cycle at 72° C. One microlitre of the reaction mix was cloned into the vector pCR® -BluntII-TOPO® using the Zero Blunt® TOPO® PCR cloning kit (Invitrogen) according to the manufacturer's instruction. Recombinant plasmids containing the FtsZ gene were purified using the Quantum Prep® Plasmid Miniprep Kit (Bio-Rad) according to the manufacturer's instructions and analysed by restriction digest with the restriction endonuclease NdeI and NcoI (both New England Biolabs) according to the manufacturer's recommendations. Digested plasmid DNA was separated on a 1% (w/v) agarose gel with TAE-buffer, stained with ethidium bromide and visualised under UV irradiation. Four bands were visible and the second smallest one (approximately 1200 bp in size) was cut out and placed into a centrifuge tube. The DNA was eluted from the gel slice by addition of 0.5 M NaCl solution and subsequent incubation for 30 min at room temperature. The gel slice was placed into a standard spin column (Bio-Rad), frozen for 10 min at −20° C. and then spun for 13 min at room temperature and 15 000× g. The flowthrough was recovered and the DNA precipitated using standard techniques as described previously (Sambrook et al., 1989). The final DNA pellet was resuspended in 20 microlitres of water.

The vector backbone was generated by digesting the plasmid pTYB1 (New England Biolabs) with the restriction enzymes NdeI and BamHI. This causes a release of a 1.4 kb fragment and a 5.8 kb fragment—the latter containing all the necessary elements for selection, replication, induction and expression. A plasmid map is shown in FIG. 3. The 5.8kbp DNA fragment was purified and processed as described in example 1.

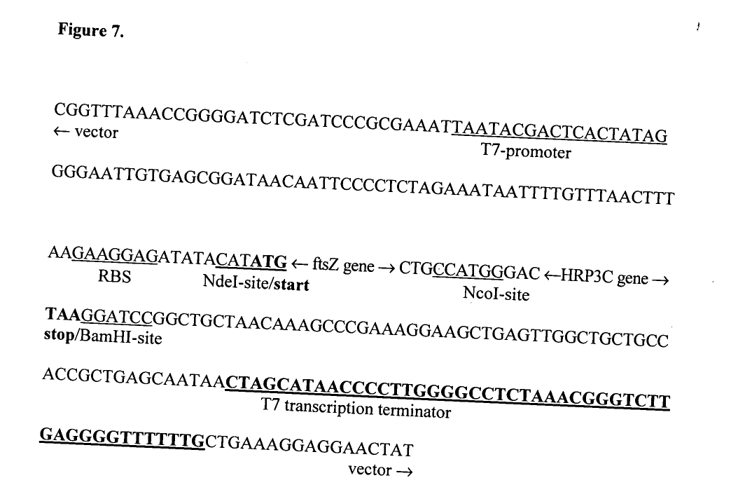

Ligation of the digested and purified HRV protease 3C DNA fragment, FtsZ DNA fragment and pTYB1 fragment were performed as follows. Fifteen nanograms of dephosphorylated pTYB1 fragment was ligated with 2.5 ng of HRV protease 3C DNA fragment and 5 ng of FtsZ DNA fragment in a 10 microliter volume using T4 DNA-ligase and ligation buffer (New England Biolabs) according to the manufacturer's recommendations. Ligation was performed overnight at 14° C. and stopped by 10 min incubation at 65° C. Five microliter of the ligation mixture was transformed into chemical-competent E. coli XL 10-Gold® cells (Stratagene) according to the manufacturer's recommendations. Recombinant plasmids containing the HRV protease 3C gene fused to the FtsZ gene were purified using the Quantum Prep® Plasmid Miniprep Kit (Bio-Rad) according to the manufacturer's instruction and checked by restriction digest with the restriction endonucleases NdeI and BamHI (New England Biolabs) according to the manufacturer's recommendations. Digested plasmid DNA was separated on a 1% (w/v) agarose gel with TAE-buffer, stained with ethidium bromide and visualised under UV irradiation. Two bands with sizes of 5.8 kb and 1.7 kb were detected corresponding to the vector backbone and the fusion between the FtsZ gene and HRV protease 3C gene, respectively. The complete insert was sequenced and the results are shown in FIG. 7. The resulting vector was termed pZTAG3CP.

(b) Expression and Purification of the Hybrid Polypeptide comprising HRV Protease 3C and GFP

The plasmid pZTAG3CP was transformed into chemical competent E. coli BL21 (DE3) cells (genotype: E. coli B F⁻ dcm ompT hsdS(r_{B}-m_{B}-) gal λ(DE3)) containing the plasmid pLysS (encoding for T7 lysozyme, a natural inhibitor of T7 RNA-polymerase). The cells were prepared and transformed by standard techniques as described in Sambrook et al. (Sambrook et al., 1989). Transformed cells were selected on LB agar plates containing 100 mg/L ampicillin and 32 mg/L chloramphenicol. After overnight incubation at 37° C. a single ampicillin and chloramphenicol-resistant colony from the transformation plate was used to inoculate 2 mL of liquid LB-medium containing 100 mg/L ampicillin and 32 mg/L chloramphenicol. After overnight incubation at 37° C. 200 microliter of this culture was used to inoculate a 20 mL culture of liquid LB-medium containing 100 mg/L ampicillin and 32 mg/L chloramphenicol. At an optical density at 610 nm (OD₆₁₀) of 0.4-0.5 isopropyl β-D-thiogalactopyranoside (IPTG) was added to the culture to give a final concentration of 1 mM. The culture was shifted from 37° C. to 30° C. and incubated for 2 h.

After incubation the cells were harvested by centrifugation at 4° C. for 2 min at 3500× g. The culture supernatant was removed and the cell pellet resuspended in 1 mL of 50 mM Tris-HCl (pH 7.5), 10% (v/v) glycerol, 1 mM PMSF and 1 mM DTT. The cells were lysed by 4×15 sec sonication using a Branson Microtip Sonicator at duty setting 80% and output level 3. The cell lysate was cleared from intact cells and large, insoluble material (such as cell membranes) by 20 min centrifugation at 4° C. and 20000× g. The pellet was discarded and the supernatant was adjusted to 1 mM MgCl₂, 20 mM CaCl₂ and 1 mM GTP. The supernatant was incubated for 30 min at 37° C. and then centrifuged for 20 min at 4° C. and 20000× g. The supernatant was discarded and the pellet containing the polymerised hybrid polypeptide was resuspended in 1 mL of 50 mM Tris-HCl (pH 7.5), 10% (v/v) glycerol, 1 mM PMSF and 1 mM DTT. The suspension was incubated for 30 min on ice and then centrifuged for 20 min at 4° C. and 20,000×g. The supernatant containing the dissociated hybrid polypeptide was recovered and the pellet resuspended in 1 mL water.

During the purification procedure samples were taken at all stages and analysed by SDS-PAGE as described by Laemmli (Laemmli, 1970) and Deutscher (Deutscher, 1990). The gel was stained with Coomassie Brilliant blue R250 using standard protocol as described in Ausubel et al. (Ausubel et al., 1994).

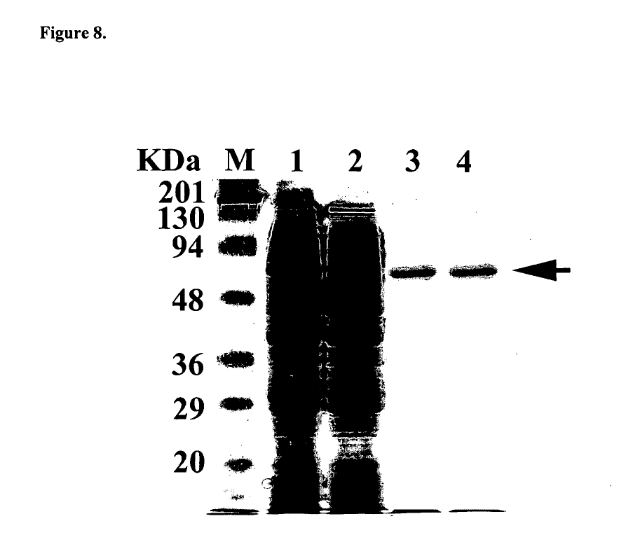

FIG. 8 shows the extent of purification of the hybrid polypeptide (HRV protease 3C/FtsZ fusion protein). Lane 1 shows that the cleared cell lysate contains a strongly overexpressed band (see arrow) of estimated molecular weight of 60 kDa, corresponding well with the combined mass of FtsZ (40.2 kDa) and HRP (20 kDa). Lane 2 shows the supernatant after the first centrifugation step and after addition of MgCl₂, CaCl₂ and GTP. Lane 3 contains a sample of the resuspended pellet after the first centrifugation step and after addition of MgCl₂, CaCl₂ and GTP and shows clearly that the hybrid polypeptide is purified in the pellet. Lane 4 contains a sample of the supernatant after ice-incubation and the second centrifugation and shows clearly that the hybrid polypeptide has been dissociated.

### EXAMPLE 3

This example describes the cleavage of a hybrid polypeptide (formed by fusion of green fluorescent protein (GFP) and E. coli FtsZ) by a protease hybrid polypeptide (formed between HRV protease 3C and E. coli FtsZ) and the subsequent removal of released E. coli FtsZ and the protease hybrid polypeptide from the released GFP (a representative polypeptide of interest).

In this example a hybrid polypeptide having a linker polypeptide comprising a protease recognition site is cleaved by a “protease hybrid polypeptide”. The self-polymerising properties of FtsZ were subsequently used to remove the protease hybrid polypeptide from the released polypeptide of interest (GFP).

A hybrid polypeptide comprising E. coli FtsZ (polymerisable polypeptide), Aequorea Victoria GFP (the polypeptide of interest) and a cleavable linker polypeptide was purified as described in Examples 1 and 2. A protease hybrid polypeptide comprising E. coli FtsZ and HRV protease 3C containing no cleavable linker was also purified as described in Examples 1 and 2. Twenty microliters each of the purified hybrid polypeptide and purified protease hybrid polypeptide were mixed and incubated for 16 h at 15° C.

Twenty microliters of the protease digest were then adjusted to 1 mM MgCl₂, 20 mM Ca Cl₂ and 1 mM GTP. The supernatant was incubated for 30 min at 37° C. and then centrifuged for 20 min at 4° C. and 20000× g. The supernatant was removed and the pellet containing the polymerised protease hybrid polypeptide was resuspended in 20 microliters of 50 mM Tris-HCl (pH 7.5) and 10% (v/v) glycerol.

During the procedure samples were taken at all stages and analysed by SDS-PAGE as described for Examples 1 and 2.

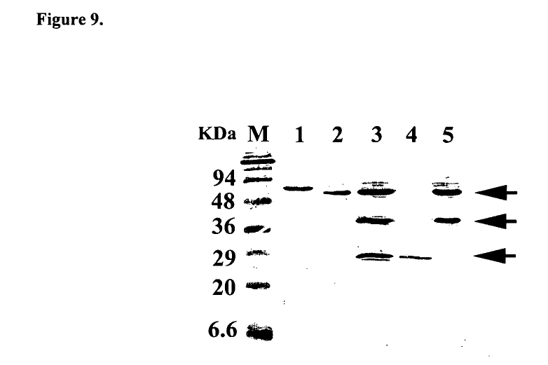

FIG. 9 shows the cleavage of the hybrid polypeptide and the removal of the protease hybrid polypeptide. Lane 1 and 2 show the purified hybrid polypeptide and protease hybrid polypeptide, respectively (see arrows). Lane 3 contains a sample of the protease treatment before the addition of MgCl₂, CaCl₂ and GTP. Lane 4 contains a sample of the supernatant after the centrifugation and addition of MgCl₂, CaCl₂ and GTP and shows the purified GFP-protein at 27 kDa (see lowest arrow). Lane 5 contains a sample of the resuspended pellet after centrifugation and addition of MgCl₂, CaCl₂ and GTP. This demonstrates that the protease hybrid polypeptide (top arrow) and the cleaved FtsZ protein (middle arrow) have been selectively polymerised and removed.

Although the invention has been described with reference to specific Examples, it will be appreciated by those skilled in the art that the invention may be embodied in many other forms.

### References

Arnold, F. H. (1991). Metal-affinity separations: a new dimension in protein processing. Bio/Technology 9, 151-156.
Ausubel, F. M., Brent, R., Kingston, R. E., Moore, D. D., Seidman, J. G., Smith, J. A. & Struhl, K. (1994) Current Protocol in Molecular Biology, John Wiley & Sons, Inc.
Bi, E. & Lutkenhaus, J. (1991). FtsZ ring structure associated with division in Escherichia coli. Nature 354, 161-164.
Bramhill, D. & Thompson, C. M. (1994). GTP-dependent polymerisation of Escherichia coli FtsZ protein to form tubules. Proc. Natl. Acad. Sci. USA. 91, 5813-5817.
Cordingley, M. G., Callahan, P. L., Sardana, V. V., Garsky, V. M. & Colonno, R. J. (1990). Substrate requirements of human rhinovirus 3C protease for peptide cleavage in vitro. J . Biol. Chem. 265, 9062-9065.
Deutscher, M. P. (1990) Guide to Protein Purification Meth. Enzymol. 182
di Gaun, C., Li, P., Riggs, P. D. & Inouye, H. (1988). Vectors that facilitate the expression and purification of foreign peptides in Escherichia coli by fusion to maltose-binding protein. Gene 67, 21-30.
Erickson, H. P., Taylor, D. W., Taylor, K. A. & Bramhill, D. (1996). Bacterial cell division protein FtsZ assembles into protofilament sheets and minirings, structural homologs of tubulin polymers. Proc. Natl. Acad. Sci. USA. 93, 519-523.
Kornberg, A. (1990). Why purify enzymes? Meth. Enzymol. 182, 1-5.
Lallo, G. D., Anderluzzi, D., Ghelardini, P. & Paolozzi, L. (1999). FtsZ dimerization in vivo. Mol. Microbiol. 32, 265-274.
Laemmli, U. K. (1970). Cleavage of structural proteins during the assembly in the head of bacteriophage T4. Nature. 227, 680-685 (1970)
Leong, L. E. -C., Walker, P. A. & Porter, A. G. (1992). Efficient expression and purification of a protease from the common cold virus, human rhinovirus type 14. J. Crystal Growth. 122, 246-252.
Löwe, J. & Amos, L. A. (1998). Crystal structure of the cell-division protein FtsZ. Nature 391, 203-206.
Löwe, J. & Amos, L. A. (1999). Tubulin-like protofilaments in Ca2+-induced FtsZ sheets. EMBO J. 18, 2364-2371.
Lu, C., Stricker, J. & Erickson, H. P. (2001) Site-specific mutations of FtsZ—effects on GTPase and in vitro assembly. BMC Microbiology. 1-7
Makrides, S. C. (1996). Strategies for achieving high-level expression of genes in Escherichia coli. Microbiol. Rev. 60, 512-538.
Mukherjee, A. & Lutkenhaus, J. (1998). Dynamic assembly of FtsZ regulated by GTP hydrolysis. EMBO J. 17, 463-469.
Mukherjee, A. & Lutkenhaus, J. (1999). Analysis of FtsZ assembly by light scattering and determination of the role of divalent metal cations. J. Bacteriol. 181, 823-832.
Ostove, S. (1990). Affinity chromatography: general methods. Meth. Enzymol. 182, 357-371.
RayChaudhuri, D. & Park, J. T. (1994). A point mutation converts Escherichia coli FtsZ septation GTPase to an ATPase. J. Biol. Chem. 269, 22941-22944.
Sambrook, J., Fritsch, E. F. & Maniatis, T. (1989). Molecular cloning, a laboratory manual, 2nd edn. New York: Cold Spring Harbor Laboratory Press.
Scheffer, D. -J., deWit, J. G., den Blaauwen, T. & Driessen, A. J. M. (2001) Substitution of a conserved aspartate allows cation-induced polymerization of FtsZ. FEBS Lett. 494, 34-37
Smith, D. B. & Johnson, K. S. (1988). Single-step purification of polypeptides expressed in Escherichia coli as fusions with glutathione S-transferase. Gene 67, 31-40.
Taylor, M. E. & Drickamer, K. (1991). Carbohydrate-recognition domains as tools for rapid purification of recombinant eucaryotic proteins. Biochem. J. 274, 575-580.
Van Dyke, M. W., Sirito, M. & Sawadogo, M. (1992). Single-step purification of bacterially expressed polypeptides containing an oligo-histidine domain. Gene 11.1, 99-104.
Xu, M. Q., Paulus, H. & Chong, S. (2000). Fusions to self-splicing inteins for protein purification. Method Enzymol 326, 376-418.
Yu, X. -C. & Margolin, W. (1997). Ca2+-mediated GTP-dependent dynamic assembly of bacterial cell division protein FtsZ into asters and polymer networks in vitro. EMBO J. 16, 5455-5463.

1. A hybrid polypeptide comprising a polypeptide of interest linked to a polymerisable polypeptide by a linker polypeptide, wherein the linker polypeptide comprises a recognition site for a proteolytic agent.

2. A hybrid polypeptide according to claim 1 wherein the proteolytic agent is selected from the following group: 3C-protease from a human rhinovirus type 14 (HRV protease 3C), thrombin, Factor Xa, enterokinase and a chemical capable of proteolytic activity.

3. A hybrid polypeptide according to claim 2 wherein the proteolytic agent is 3C-protease from a human rhinovirus type 14 (HRV protease 3C).

4. A hybrid polypeptide according to claim 1 wherein the recognition site comprises an amino acid sequence selected from the following group:
Leu-Glu-Val-Leu-Phe-Gln-Gly-Pro, Leu-Val-Pro-Arg-Gly-Ser, Ile-Glu-Gly-Arg and Asp-Asp-Asp-Asp-Lys.

5. A hybrid polypeptide according to claim 2 wherein the chemical capable of proteolytic activity is cyanogen bromide.

6. A hybrid polypeptide according to any one of claims 1 to 5 wherein the linker polypeptide is encoded by a polynucleotide comprising a cloning site.

7. A hybrid polypeptide according to claim 6 wherein the cloning site is a multiple cloning site.

8. A hybrid polypeptide according to any one of claims 1 to 7 wherein the linker polypeptide comprises a spacer polypeptide of sufficient length to allow or enhance cleavage of the polypeptide of interest from the polymerisable polypeptide, or to avoid unfavourable steric interference between the polypeptide of interest and the polymerisable polypeptide.

9. A hybrid polypeptide according to claim 1 wherein the polypeptides are linked by antibody interaction.

10. A hybrid polypeptide according to claim 9 wherein the antibody interaction is achieved by a process comprising attaching an antibody specific for the polymerisable polypeptide to the polypeptide of interest.

11. A hybrid polypeptide according to claim 9 wherein the antibody interaction is achieved by a process comprising attaching an antibody specific for the polypeptide of interest to the polymerisable polypeptide.

12. A hybrid polypeptide according to claim 9 wherein the antibody interaction is achieved using a bi-specific antibody directed to both the polypeptide of interest and the polymerisable polypeptide.

13. A hybrid polypeptide according to any one of claims 1 to 12 wherein the polymerisable polypeptide is a polypeptide that naturally polymerises with itself.

14. A hybrid polypeptide according to claim 13 the polymerisable polypeptide is tubulin or actin.

15. A hybrid polypeptide according to claim 13 wherein the polymerisable polypeptide is an FtsZ protein or a variant thereof.

16. A hybrid polypeptide according to claim 15 wherein the polymerisable peptide is E. coli FtsZ protein or a variant thereof.

17. A hybrid polypeptide according to claim 16 wherein the variant E. coli FtsZ protein comprises replacement of the aspartate residue at position 212 of the protein with a cysteine or asparagine residue.

18. A hybrid polypeptide according to claim 16 wherein the variant FtsZ comprises a mutation selected from one of the following: replacement of alanine by threonine at position 70, replacement of aspartate by alanine at position 209 and replacement of aspartate by alanine at position 269.

19. A hybrid polypeptide according to any one of claims 1 to 18 wherein the polymerisable polypeptide requires an intermediary polypeptide or other molecule in order to polymerise.

20. A hybrid polypeptide according to any one of claims 1 to 19 wherein the polypeptide of interest is of prokaryotic origin.

21. A hybrid polypeptide according to any one of claims 1 to 19 wherein the polypeptide of interest is of eukaryotic origin.

22. A hybrid polypeptide according to any one of claims 1 to 21 wherein the polypeptide of interest is selected from the group comprising: an endonuclease, a methylase, an oxidoreductase, a transferase, a hydrolase, a lysase, an isomerase, a ligase, a storage polypeptide, a ferritin, an ovalbumin, a transport protein, haemoglobin, serum albumin or ceruloplasmin, an antigen, an antigenic determinant for use in the preparation of vaccines or diagnostic agents, a protective protein, a defence protein, thrombin, fibrinogen, binding proteins, antibodies, immunoglobulins, a human growth hormone, somatostatin, prolactin, estrone, progesterone, melanocyte, thyrotropin, calcitonin, gonadotropin, insulin, a hormone identified as being involved in the immune system, interleukin 1, interleukin 2, colony simulating factor, macrophage-activating factor, interferon, a structural element, collagen, elastin, alpha-keratin, glyco-protein, virus-protein and muca-protein.

23. A hybrid polypeptide according to claim 22 wherein the polypeptide of interest is a protease.

24. A hybrid polypeptide according to claim 23 wherein the protease is 3C-protease from human rhinovirus type 14 (HRV protease 3C).

25. A hybrid polypeptide according to any one of claims 1 to 23 wherein the polypeptide of interest is a synthetic polypeptide.

26. A method of sequestering and/or purifying a polypeptide of interest comprising the step of polymerising a hybrid polypeptide which hybrid polypeptide comprises the polypeptide of interest linked to a polymerisable polypeptide.

27. A method according to claim 26 wherein the polypeptide of interest is linked to the polymerisable polypeptide by fusing the polypeptide of interest directly to the polymerisable polypeptide.

28. A method according to claim 26 wherein the polypeptide of interest is linked to the polymerisable polypeptide by a linker polypeptide.

29. A method according to any one of claims 26 to 28 wherein the hybrid polypeptide is produced in vivo.

30. A method according to claim 26 wherein the hybrid polypeptide is a polypeptide according to any one of claims 1 to 25.

31. A method according to any one of claims 26 to 30 wherein polymerisation is performed under controlled chemical and/or physical conditions.

32. A method according to any one of claims 26 to 31 wherein the polymerisable polypeptide is polymerised by the addition of an agent which induces polymerisation.

33. A method according to claim 32 wherein the polymerisation inducing agent is GTP, ATP and/or a cation.

34. A method according to claim 33 wherein the cation is selected from the following group: magnesium, calcium, nickel, cobalt, zinc and manganese.

35. A method according to claim 31 wherein the polymerisable polypeptide is polymerised by a change in temperature.

36. A method according to any one of claims 26 to 35 wherein the polymerised hybrid polypeptide is purified by a first purification step and wherein the first purification step may be the only purification step or may be followed by further purification steps.

37. A method according to claim 36 wherein the first purification step purifies the polymerised hybrid polypeptide by physical techniques discriminating on the basis of size and/or weight.

38. A method according to claim 37 wherein the polymerised hybrid polypeptide is purified by centrifugation, differential sedimentation, filtration, dialysis and/or flow sorting such that the polymerised hybrid polypeptide is isolated.

39. A method according to claim 38 wherein after the first purification step the polymerised hybrid polypeptide is dissociated.

40. A method according to claim 39 wherein dissociation is achieved by removal of the agent which induces polymerisation and/or incubation of the polymerised hybrid polypeptide at a suitable temperature.

41. A method according to claim 39 or claim 40 wherein the dissociated hybrid polypeptide is purified by a second purification step.

42. A method according to claim 41 wherein the second purification step comprises purification of the hybrid polypeptide on the basis of size and/or weight.

43. A method according to claim 41 wherein polymerisation, dissociation and purification of the polymerisable hybrid polypeptide are repeated such that substances larger and smaller than the hybrid polypeptide are removed.

44. A method according to any one of claims 26 to 43 wherein the polymerisable polypeptide is cleaved from the polypeptide of interest by a proteolytic agent.

45. A method according to claim 44 wherein cleavage by the proteolytic agent does not substantially interfere with the biological or chemical activity of the polypeptide of interest or the polymerisable polypeptide.

46. A method according to claim 44 or claim 45 wherein the proteolytic agent is a protease.

47. A method according to claim 46 wherein the protease is linked to a polymerisable polypeptide to form a “protease hybrid polypeptide”.

48. A method according to claim 47 wherein the polymerisable polypeptide to which the protease is linked is identical to the polymerisable polypeptide to which the polypeptide of interest is linked, or is a variant thereof.

49. A method according to claim 47 or claim 48 wherein after cleavage of the polypeptide of interest from the polymerisable polypeptide, the protease hybrid polypeptide is polymerised.

50. A method according to claim 49 wherein the polypeptide of interest is purified from the polymerised protease hybrid polypeptide.

51. A method according to any one of claims 44 to 46 wherein the proteolytic agent is fused to the hybrid polypeptide.

52. A method according to any one of claims 44 to 51 wherein the polymerisable polypeptide released after cleavage from the polypeptide of interest is polymerised.

53. A method according to claim 52 wherein the polymerised polymerisable polypeptide is removed from the polypeptide of interest by a method which discriminates on the basis of size and/or weight.

54. A method according to any one of claims 46 to 53 wherein the protease is 3C-protease from a human rhinovirustype 14 (HV protease 3C).

55. A method according to any one of claims 26 to 54 wherein the hybrid polypeptide is linked to a support.

56. A method according to claim 55 wherein the support comprises a polymerisable polypeptide.

57. A method according to claim 56 wherein the support polymerisable polypeptide comprises a polymerisable polypeptide identical to the hybrid polypeptide, or a variant thereof.

58. A hybrid nucleic acid comprising a nucleic acid encoding a hybrid polypeptide according to any one of claims 1 to 25.

59. A library comprising a plurality of hybrid nucleic acids according to claim 58.

60. A vector comprising a hybrid nucleic acid according to claim 58.

61. A library of vectors comprising vectors according to claim 60.

62. A cell transformed or transfected with a hybrid nucleic acid according to claim 58, a library according to claim 59, a vector according to claim 60, or a library of vectors according to claim 61.

63. Cells transformed or transfected with a library according to claim 59 or 61.

64. A library comprising a plurality of hybrid polypeptides according to any one of claims 1 to 25.

65. Use of a hybrid nucleic acid according to claim 58, a library according to claim 59, a vector according to claim 60, or a library of vectors according to claim 61 in a method of sequestering and/or purifying a polypeptide of interest.

66. A polypeptide of interest when purified by a method according to any one of claims 26 to 57.

67. A library of polypeptides of interest according to claim 66.

68. A method of purifying a polypeptide of interest comprising:
(a) expressing the hybrid nucleic acid of claim 58 in a cell to produce a hybrid polypeptide comprising the polypeptide of interest and a polymerisable polypeptide;
(b) polymerising the hybrid polypeptide;
(c) purifying the polymerised hybrid polypeptide;
(d) cleaving the polypeptide of interest from the polymerisable polypeptide; and
(e) purifying the polypeptide of interest.

69. A method according to claim 68 wherein the polypeptide of interest is cleaved from the polymerisable polypeptide by a protease which protease is itself linked to a polymerisable polypeptide to form a protease hybrid polypeptide.

70. A method according to claim 69 wherein after cleavage, the polymerisable polypeptide linked to the protease is polymerised and the polypeptide of interest is purified by removal of the polymerised protease hybrid polypeptide.


SEQUENCE LISTING (12 sequences)

SEQ ID NO: 1 - DNA, 714 bp, Aequorea victoria
atgagtaaaggagaagaacttttcactggagttgtcccaattcttgttgaattagatggcgatgttaatgggcaaaaattctctgtcagtggagagggtgaaggtgatgcaacatacggaaaacttacccttaaatttatttgcactactgggaagctacctgttccatggccaacacttgtcactactttcgcgtatggtcttcaatgctttgcgagatacccagatcatatgaaacagcatgactttttcaagagtgccatgcccgaaggttatgtacaggaaagaactatattttacaaagatgacgggaactacaagacacgtgctgaagtcaagtttgaaggtgatacccttgttaatagaatcgagttaaaaggtattgattttaaagaagatggaaacattcttggacacaaaatggaatacaactataactcacataatgtatacatcatggcagacaaaccaaagaatggaatcaaagttaacttcaaaattagacacaacattaaagatggaagcgttcaattagcagaccattatcaacaaaatactccaattggcgatggccctgtccttttaccagacaaccattacctgtccacacaatctgccctttccaaagatcccaacgaaaagagagatcacatgatccttcttgagtttgtaacagctgctgggattacacatggcatggatgaactatacaaa

SEQ ID NO: 2 - DNA, 1149 bp, Escherichia coli
atgtttgaaccaatggaacttaccaatgacgcggtgattaaagtcatcggcgtcggcggcggcggcggtaatgctgttgaacacatggtgcgcgagcgcattgaaggtgttgaattcttcgcggtaaataccgatgcacaagcgctgcgtaaaacagcggttggacagacgattcaaatcggtagcggtatcaccaaaggactgggcgctggcgctaatccagaagttggccgcaatgcggctgatgaggatcgcgatgcattgcgtgcggcgctggaaggtgcagacatggtctttattgctgcgggtatgggtggtggtaccggtacaggtgcggcaccagtcgtcgctgaagtggcaaaagatttgggtatcctgaccgttgctgtcgtcactaagcctttcaactttgaaggcaagaagcgtatggcattcgcggagcaggggatcactgaactgtccaagcatgtgaactctctgatcactatcccgaacgacaaactgctgaaagttctgggccgcggtatctccctgctggatgcgtttggcgcagcgaacgatgtactgaaaggcgctgtgcaaggtatcgctgaactgattactcgtccgggtttgatgaacgtggactttgcagacgtacgcaccgtaatgtctgagatgggccacgcaatgatgggttctggcgtggcgagcggtgaagaccgtgcggaagaagctgctgaaatggctatctcttctccgctgctggaagatatcgacctgtctggcgcgcgcggcgtgctggttaacatcacggcgggcttcgacctgcgtctggatgagttcgaaacggtaggtaacaccatccgtgcatttgcttccgacaacgcgactgtggttatcggtacttctcttgacccggatatgaatgacgagctgcgcgtaaccgttgttgcgacaggtatcggcatggacaaacgtcctgaaatcactctggtgaccaataagcaggttcagcagccagtgatggatcgctaccagcagcatgggatggctccgctgacccaagagcagaagccggttgctaaagtcgtgaatgacaatgcgccgcaaactgcgaaagagccggattatctggatatcccagcattcctgcgtaagcaagctgat

SEQ ID NO: 3 - DNA, 546 bp, Human rhinovirus
ggaccaaacacagaatttgcactatccctgttaaggaaaaacataatgactataacaacctcaaagggagagttcacagggttaggcatacatgatcgtgtctgtgtgatacccacacacgcacagcctggtgatgatgtactagtgaatggtcagaaaattagagttaaggataagtacaaattagtagatccagagaacattaatctagagcttacagtgttgactttagatagaaatgaaaaattcagagatatcaggggatttatatcagaagatctagaaggtgtggatgccactttggtagtacattcaaataactttaccaacactatcttagaagttggccctgtaacaatggcaggacttattaatttgagtagcacccccactaacagaatgattcgttatgattatgcaacaaaaactgggcagtgtggaggtgtgctgtgtgctactggtaagatctttggtattcatgttggcggtaatggaagacaaggattttcagctcaacttaaaaaacaatattttgtagagaaacaa

SEQ ID NO: 4 - DNA, 27 bp, artificial sequence
  Polymerase chain reaction oligonucleotide primer
atcatgagtaaaggagaagaacttttc

SEQ ID NO: 5 - DNA, 29 bp, artificial sequence
  Polymerase chain reaction oligonucleotide primer
aggatccttatttgtatagttcatccatg

SEQ ID NO: 6 - DNA, 24 bp, artificial sequence
  OTHER INFORMATION Polymerase chain reaction oligonucleotide primer
ggcatatgtttgaaccaatggaac

SEQ ID NO: 7 - DNA, 27 bp, artificial sequence
  Polymerase chain reaction oligonucleotide primer
gtccatgggcccttgaaatagtacttc

SEQ ID NO: 8 - DNA, 43 bp, artificial sequence
  Polymerase chain reaction oligonucleotide primer
gggcccttgaaatagtacttctagatcagcttgcttacgcagg

SEQ ID NO: 9 - DNA, 27 bp, artificial sequence
  Polymerase chain reaction oligonucleotide primer
cgccatgggaccaaacacagaatttgc

SEQ ID NO: 10 - DNA, 32 bp, artificial sequence
  Polymerase chain reaction oligonucleotide primer
gcggatccctattgtttctctacaaaatattg

SEQ ID NO: 11 - DNA, 24 bp, artificial sequence
  Polymerase chain reaction oligonucleotide primer
ggcatatgtttgaaccaatggaac

SEQ ID NO: 12 - DNA, 25 bp, artificial sequence
  Polymerase chain reaction oligonucleotide primer
cgccatggcagcttgcttacgcagg